(19) United States
(12) Patent Application Publication
(10) Pub. No.: US 2026/0241018 A1
(43) Pub. Date: Aug. 20, 2026

(54) ANTI-5T4 ANTIBODIES AND USES THEREOF

(71) Applicant: Biocytogen Pharmaceuticals (Beijing) Co., Ltd., Beijing (CN)

(72) Inventors: Yanfei Han, Beijing (CN); Zhenyan Han, Beijing (CN); Chengzhang Shang, Beijing (CN); Yuelei Shen, Beijing (CN)

(21) Appl. No.: 19/160,615

(22) PCT Filed: Mar. 8, 2024
(86) PCT No.: PCT/CN2024/080719

§ 371 Date: Aug. 28, 2025

(30) Foreign Application Priority Data
Mar. 8, 2023 (WO) PCT/CN2023/080242

(51) Int. Cl. A61K 47/68; A61P 35/00; C07K 16/28; C07K 16/30

(52) U.S. Cl. CPC A61K 47/68031; A61K 47/6851; A61P 35/00; C07K 16/2818; C07K 16/2827; C07K 16/2878; C07K 16/30; C07K 2317/14; C07K 2317/24; C07K 2317/31; C07K 2317/33; C07K 2317/565; C07K 2317/622; C07K 2317/72; C07K 2317/732; C07K 2317/734; C07K 2317/77

(57) ABSTRACT

Provided are anti-5T4 (5T4 oncofoetal antigen) antibodies, antigen-binding fragments thereof, antibody-drug conjugate (ADC) derived therefrom, and the uses thereof.

### CLAIM OF PRIORITY

This application claims priority to PCT/CN2023/080242, filed on Mar. 8, 2023. The entire contents of the foregoing application are incorporated herein by reference.

### TECHNICAL FIELD

This disclosure relates to anti-5T4 (5T4 oncofoetal antigen) antibodies, antigen-binding fragments thereof, antibody-drug conjugate (ADC) derived therefrom, and the uses thereof.

### BACKGROUND

Trophoblast glycoprotein, also known as 5T4, TPBG, Wnt-Activated Inhibitory Factor 1 or WAIF1, is a human protein encoded by a 5T4 gene. The 5T4 protein is expressed by many different cancers but rarely in normal adult tissues. 5T4 is an antagonist of Wnt/β-catenin signalling pathway.

Considering the important role of 5T4 in tumors, there is a need to develop a therapeutic agent targeting 5T4.

### SUMMARY

This disclosure relates to anti-5T4 antibodies, antigen-binding fragment thereof, and the uses thereof.

In one aspect, the disclosure is related to an antibody or antigen-binding fragment thereof that binds to 5T4 (5T4 oncofoetal antigen) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 1-3, respectively; and
(12) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 1-3, respectively.

In some embodiments, the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 1-3, respectively, according to Kabat definition.

In some embodiments, the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 1-3, respectively, according to Chothia definition.

In some embodiments, the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 1-3, respectively, according to Chothia definition.

In some embodiments, the antibody or antigen-binding fragment thereof specifically binds to human, mouse, monkey, or dog 5T4.

In some embodiments, the antibody or antigen-binding fragment thereof is a human or humanized antibody or antigen-binding fragment thereof, a single-chain variable fragment (scFv), a one-armed antibody, and/or a multi-specific antibody (e.g., a bispecific antibody).

In some embodiments, the antibody or antigen-binding fragment thereof is a human IgG1 antibody or antigen-binding fragment thereof or a human IgG4 antibody or antigen-binding fragment thereof.

In one aspect, the disclosure is related to a nucleic acid comprising a polynucleotide encoding a polypeptide comprising:
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 41 binds to 5T4;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 42 binds to 5T4;
an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 43 binds to 5T4;
an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 44 binds to 5T4;
an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 45 binds to 5T4;
an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 46 binds to 5T4;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 47 binds to 5T4;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 40 binds to 5T4;
an immunoglobulin heavy chain or a fragment thereof comprising a VH comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 40 binds to 5T4;
an immunoglobulin heavy chain or a fragment thereof comprising a VH comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 40 binds to 5T4;
an immunoglobulin light chain or a fragment thereof comprising a VL comprising CDRs 1, 2, and 3 comprising the amino acid sequences set forth in SEQ ID NOs: 31-33, respectively, and wherein the VL, when paired with a VH comprising the amino acid sequence set forth in SEQ ID NO: 40 binds to 5T4;
an immunoglobulin heavy chain or a fragment thereof comprising a VH comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 40 binds to 5T4; or
an immunoglobulin heavy chain or a fragment thereof comprising a VH comprising complementarity determining regions (CDRs) 1, 2, and 3 comprising the amino acid sequences set forth in SEQ ID NOs: 37-39, respectively, and wherein the VH, when paired with a light chain variable region (VL) comprising the amino acid sequence set forth in SEQ ID NO: 40 binds to 5T4.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4-6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7-9, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10-12, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13-15, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16-18, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19-21, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 22-24, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25-27, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 28-30, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 31-33, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 34-36, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 37-39, respectively.

In some embodiments, the VH when paired with a VL specifically binds to human, mouse, monkey, or dog 5T4, or the VL when paired with a VH specifically binds to human, mouse, monkey, or dog 5T4.

In some embodiments, the immunoglobulin heavy chain or the fragment thereof is a human or humanized immunoglobulin heavy chain or a fragment thereof (e.g., a human IgG1 heavy chain or a fragment thereof; or a human IgG4 heavy chain or a fragment thereof), and the immunoglobulin light chain or the fragment thereof is a human or humanized immunoglobulin light chain or a fragment thereof.

In some embodiments, the nucleic acid encodes a single-chain variable fragment (scFv), a one-armed antibody, a multi-specific antibody (e.g., a bispecific antibody), or a chimeric antigen receptor (CAR).

In some embodiments, the nucleic acid is cDNA.

In one aspect, the disclosure is related to a vector comprising one or more of the nucleic acids described herein.

In one aspect, the disclosure is related to a vector comprising two of the nucleic acids described herein, wherein the vector encodes the VL region and the VH region that together bind to 5T4.

In one aspect, the disclosure is related to a pair of vectors, wherein each vector comprises one of the nucleic acids described herein, wherein together the pair of vectors encodes the VL region and the VH region that together bind to 5T4.

In one aspect, the disclosure is related to a cell comprising the vector described herein, or the pair of vectors described herein.

In some embodiments, the cell is a CHO cell.

In one aspect, the disclosure is related to a cell comprising one or more of the nucleic acids described herein.

In one aspect, the disclosure is related to a cell comprising two of the nucleic acids described herein.

In some embodiments, the two nucleic acids together encode the VL region and the VH region that together bind to 5T4.

In one aspect, the disclosure is related to a method of producing an antibody or an antigen-binding fragment thereof, the method comprising culturing the cell described herein under conditions sufficient for the cell to produce the antibody or the antigen-binding fragment; and collecting the antibody or the antigen-binding fragment produced by the cell.

In one aspect, the disclosure is related to an antibody or antigen-binding fragment thereof that binds to 5T4 comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
the selected VH sequence is SEQ ID NO: 41, and the selected VL sequence is SEQ ID NO: 40;
the selected VH sequence is SEQ ID NO: 42, and the selected VL sequence is SEQ ID NO: 40;
the selected VH sequence is SEQ ID NO: 43, and the selected VL sequence is SEQ ID NO: 40;
the selected VH sequence is SEQ ID NO: 44, and the selected VL sequence is SEQ ID NO: 40;
the selected VH sequence is SEQ ID NO: 45, and the selected VL sequence is SEQ ID NO: 40;
the selected VH sequence is SEQ ID NO: 46, and the selected VL sequence is SEQ ID NO: 40; and
the selected VH sequence is SEQ ID NO: 47, and the selected VL sequence is SEQ ID NO: 40.

In some embodiments, the VH comprises the sequence of SEQ ID NO: 41 and the VL comprises the sequence of SEQ ID NO: 40.

In some embodiments, the VH comprises the sequence of SEQ ID NO: 42 and the VL comprises the sequence of SEQ ID NO: 40.

In some embodiments, the VH comprises the sequence of SEQ ID NO: 43 and the VL comprises the sequence of SEQ ID NO: 40.

In some embodiments, the VH comprises the sequence of SEQ ID NO: 44 and the VL comprises the sequence of SEQ ID NO: 40.

In some embodiments, the VH comprises the sequence of SEQ ID NO: 45 and the VL comprises the sequence of SEQ ID NO: 40.

In some embodiments, the VH comprises the sequence of SEQ ID NO: 46 and the VL comprises the sequence of SEQ ID NO: 40.

In some embodiments, the VH comprises the sequence of SEQ ID NO: 47 and the VL comprises the sequence of SEQ ID NO: 40.

In one aspect, the disclosure is related to an antibody or antigen-binding fragment thereof that binds to 5T4 comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
the selected VH sequence is SEQ ID NO: 41, and the selected VL sequence is SEQ ID NO: 40;
the selected VH sequence is SEQ ID NO: 42, and the selected VL sequence is SEQ ID NO: 40;
the selected VH sequence is SEQ ID NO: 43, and the selected VL sequence is SEQ ID NO: 40;
the selected VH sequence is SEQ ID NO: 44, and the selected VL sequence is SEQ ID NO: 40;
the selected VH sequence is SEQ ID NO: 45, and the selected VL sequence is SEQ ID NO: 40;
the selected VH sequence is SEQ ID NO: 46, and the selected VL sequence is SEQ ID NO: 40; and
the selected VH sequence is SEQ ID NO: 47, and the selected VL sequence is SEQ ID NO: 40.

In some embodiments, the antibody or antigen-binding fragment thereof specifically binds to human, mouse, monkey, or dog 5T4.

In some embodiments, the antibody or antigen-binding fragment thereof is a human or humanized antibody or antigen-binding fragment thereof, a single-chain variable fragment (scFv), a one-armed antibody, and/or a multi-specific antibody (e.g., a bispecific antibody).

In some embodiments, the antibody or antigen-binding fragment is a human IgG1 antibody or antigen-binding fragment thereof or a human IgG4 antibody or antigen-binding fragment thereof.

In one aspect, the disclosure is related to an antibody or antigen-binding fragment thereof that cross-competes with the antibody or antigen-binding fragment thereof described herein.

In some embodiments, the antibody or antigen-binding fragment thereof comprises a fragment crystallizable region (Fc region).

In some embodiments, the Fc region has increased complement-dependent cytotoxicity (CDC) or antibody-dependent cellular cytotoxicity (ADCC).

In one aspect, the disclosure is related to a chimeric antigen receptor (CAR) comprising the antibody or antigen-binding fragment thereof described herein.

In one aspect, the disclosure is related to an antibody-drug conjugate comprising the antibody or antigen-binding fragment thereof described herein covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof described herein, the CAR described herein or the antibody-drug conjugate described herein, to the subject.

In some embodiments, the subject has a solid tumor.

In some embodiments, the cancer is colorectal cancer, gastric cancer, ovarian cancer, lung cancer, head and neck cancer, or pancreatic cancer.

In some embodiments, the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

In one aspect, the disclosure is related to a method of decreasing the rate of tumor growth, the method comprising contacting a tumor cell with an effective amount of a composition comprising an antibody or antigen-binding fragment thereof described herein, the CAR described herein or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, the CAR described herein or the antibody-drug conjugate described herein.

In one aspect, the disclosure is related to a method of increasing immune response in a subject, the method comprising administering to the subject an effective amount of a composition comprising the antibody or antigen-binding fragment thereof described herein.

In one aspect, the disclosure is related to a pharmaceutical composition comprising the antibody or antigen-binding fragment thereof described herein, and a pharmaceutically acceptable carrier.

In one aspect, the disclosure is related to a pharmaceutical composition comprising the antibody drug conjugate described herein, and a pharmaceutically acceptable carrier.

In some embodiments, the drug-to-antibody ratio (DAR) is about 4.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., 5T4) to at least one second target molecule on the surface of a mammalian cell (e.g., a human T-cell).

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., 5T4) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a 5T4 molecule may be referred to as a 5T4-specific antibody or an anti-5T4 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 lists heavy chain variable region and light chain variable region CDR sequences of anti-5T4 antibodies as defined by Kabat definition.

FIG. 2 lists heavy chain variable region and light chain variable region CDR sequences of anti-5T4 antibodies as defined by Chothia definition.

FIG. 3 lists amino acid sequences of heavy chain variable regions and light chain variable regions of anti-5T4 antibodies.

FIG. 4 shows the average tumor volume in different groups of B-NDG mice that were injected with NUGC-4 cells, and were treated with 3 mg/kg 32G1-ADC (G2) or 3 mg/kg PF06263507-ADC (G3). PBS was injected as a control (G1).

FIG. 5 shows the average tumor volumes in different groups of B-NDG mice that were engrafted with patient-derived lung tumor fragments (2 mm×2 mm×2 mm), and were treated with phosphate buffer saline (PBS) or ADCs.

FIG. 6A shows the binding between NCI-H226 cells and the anti-5T4 antibody 32G1, as determined by flow cytometry.

FIG. 6B shows the binding between NCI-H2030 cells and the anti-5T4 antibody 32G1, as determined by flow cytometry.

FIG. 6C shows the binding between HCC827 cells and the anti-5T4 antibody 32G1, as determined by flow cytometry.

FIG. 7 lists selected amino acid sequences discussed in the disclosure.

### DETAILED DESCRIPTION

The present disclosure provides examples of antibodies, antigen-binding fragment thereof, that bind to 5T4.

### 5T4

The 5T4 protein is expressed by many different cancers but rarely in normal adult tissues. 5T4 molecules are 72 kD, heavily N-glycosylated proteins with several leucine-rich repeats which are often associated with protein-protein interactions. 5T4 expression is associated with the directional movement of cells through epithelial mesenchymal transition, potentiation of CXCL12/CXCR4 chemotaxis and inhibition of canonical Wnt/beta-catenin while favoring non-canonical pathway signaling; all processes which help drive the spread of cancer cells. The selective pattern of 5T4 tumor expression, association with a tumor-initiating phenotype plus a mechanistic involvement with cancer spread have underwritten the clinical development of different immunotherapeutic strategies including a vaccine, a tumor-targeted superantigen and an antibody drug conjugate.

5T4 Trophoblast glycoprotein was discovered in the context of trying to identify shared cell surface molecules that may function to allow survival of the foetus as a semi-allograft in the mother, or a tumour in its host. The rationale was that such shared expression would reflect common functions relevant to growth, invasion or altered immune surveillance in the host. Murine monoclonal antibodies were raised against purified glycoproteins from trophoblast membrane preparations from term human placenta and initially screened against different cancer cell lines and human peripheral blood mononuclear cells. Further screening using the 5T4 monoclonal antibody (mAb) by immunohistochemistry indicated the antigen was expressed by many different cancers but with a restricted normal tissue distribution. A series of biochemical and genomic studies identified 5T4 molecules as N-glycosylated proteins with an apparent molecular size of 72 kD and are encoded on chromosome 6q14-15. The human gene encodes a 42 kD transmembrane protein core which contains several leucine-rich repeats (LRRs) that are associated with protein-protein interactions of a functionally diverse set of molecules. The extracellular part of the molecule has several LRRs in two domains separated by a short hydrophilic sequence; there is a transmembrane domain and a short cytoplasmic sequence. Importantly, the 5T4-specific mAb recognises a conformationally dependent epitope that relies on the integrity of the intramolecular S—S bonds and the indirect presence of the complex N-linked glycosylation. When human 5T4 was overexpressed in murine fibroblasts, the cells became more spindle shaped and had reduced adherence while in normal epithelial cells there was E-Cadherin down-regulation, increased motility and cytoskeletal disruption.

Immunohistochemistry (IHC) of frozen sections established that the 5T4-specific monoclonal antibody detected antigen expression by many different types of carcinoma but demonstrated only low-level expression in some normal adult tissue epithelia. Importantly, 5T4 was expressed in many different primary and metastatic cancers, frequently at high levels; in some cases, there was an additional stromal expression. Linking a role for 5T4 in tumour development and spread, it has been shown that 5T4 expression in colorectal, gastric and ovarian cancers correlates with poorer clinical outcome.

Several “5T4-specific” antibodies made against specific peptides or sequences of the 5T4 molecule have become available commercially. However, the specificity may not be identical to the original mAb as the precise epitopes have often not been identified and could include parts of the 5T4 molecule which contains leucine-rich repeats which are shared by a large number of proteins of diverse function and expression. However, for antibody-targeted therapies, the individual property of the reagent in the context of live tissues is the only significant relevant property.

Overexpression of 5T4 in normal murine epithelial cells is associated with E-cadherin down-regulation which is a key component of EMT; this occurs during embryonic development and is important for the metastatic spread of epithelial tumours. 5T4 was shown to be a marker of the early differentiation of mouse and human embryonic stem (ES) cell, and this process involves an E- to N-cadherin switch, upregulation of E-cadherin repressor molecules (Snail and Slug proteins), increased matrix metalloproteinase (MMP-2 and MMP-9) activity and motility, all classic EMT features. Undifferentiated knock-out (KO) E-Cadherin ES cells constitutively express cell surface 5T4 molecules while antibody induced down-regulation of E-Cadherin in ES cells induced 5T4 membrane expression, increased motility, altered actin cytoskeleton arrangement and a mesenchymal phenotype. These observations are consistent with E-cadherin somehow preventing 5T4 cell surface expression; the possible mechanism being through stabilisation of cortical actin cytoskeletal organisation. Co-expression of 5T4 and factors involved in the epithelial-to-mesenchymal transition was also observed in undifferentiated but not in differentiated lung tumour cells.

5T4 molecules have been shown to be involved in the functional expression of CXCR4 at the cell surface in some embryonic and tumour cells. Both CXCL12 and CXCR4 expression have been associated with tumourigenesis in many cancers, and it is believed that CXCR4 expression facilitates the spread to tissues that highly express CXCL12 including lung, liver, lymph nodes and bone marrow. 5T4 is expressed by putative leukaemia initiating cells in BCP-ALL, and these cells show the associated property of CXCL12/CXCR4 chemotaxis. 5T4-positive leukaemia-initiating cells are likely attracted by CXCL12 produced by extramedullary sites where there is decreased therapeutic bioavailability leading to disease relapse following treatment.

Wnt protein intracellular signalling is a central component of many aspects of cellular regulation critical to normal development, homoeostasis and regeneration, while misregulation can lead to disease, including cancer. There are two pathways, the most characterised being the canonical Wnt/β-catenin pathway while non-canonical Wnt signalling through a cell autonomous planar cell polarity (PCP) type pathway can drive the modulation of actin and microtubular skeletons facilitating cell movement in development of cancer. 5T4 has been shown to interfere with Wnt/β-catenin signalling and concomitantly activate non-canonical Wnt pathways. 5T4 binds to the Wnt co-receptor LRP6 and inhibits Wnt-induced LRP6 internalisation into endocytic vesicles, a process that is required for pathway activation thereby modulating Wnt/β-catenin signalling by regulating LRP6 subcellular localisation. In addition, 5T4 enhances β-catenin-independent Wnt signalling in promoting a non-canonical function of Dickkopf1. These results suggest that 5T4 facilitates pathway selection in Wnt-receiving cells, inhibiting Wnt/β-catenin canonical while concomitantly activating the non-canonical Wnt signalling pathway associated with increased motility. 1.8 Å resolution crystal of the extracellular domain of 5T4 and associated cell biological studies have provided a structural basis for inhibition of Wnt/β-catenin signalling.

The selective pattern of 5T4 tumour expression, association with a tumour-initiating phenotype plus a mechanistic involvement with cancer spread have underwritten the development of several different immunotherapeutic strategies.

A detailed review of 5T4 and its functions can be found in Stern, Peter L., and Richard Harrop. “5T4 oncofoetal antigen: an attractive target for immune intervention in cancer.” Cancer Immunology, Immunotherapy 66.4 (2017): 415-426; and Harrop, Richard, Eric O'Neill, and Peter L. Stern. “Cancer stem cell mobilization and therapeutic targeting of the 5T4 oncofetal antigen.” Therapeutic advances in vaccines and immunotherapy 7 (2019): 2515135518821623; each of which is incorporated by reference in its entirety.

The present disclosure provides several anti-5T4 antibodies, antigen-binding fragments thereof, and methods of using these anti-5T4 antibodies and antigen-binding fragments to inhibit tumor growth and treat cancers.

### Antibodies and Antigen Binding Fragments

The present disclosure provides anti-5T4 antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (Jan.1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting 5T4 and an addition antigen (e.g., OX40, CD3, 4-1BB, CD314, CD47, PD-1, CTLA4, CD40 or PDL1).

### Anti-5T4 Antibodies and Antigen-Binding Fragments

The disclosure provides antibodies and antigen-binding fragments thereof that specifically bind to 5T4 (e.g., human 5T4). The antibodies and antigen-binding fragments described herein are capable of binding to 5T4. These antibodies can be agonists or antagonists. In some embodiments, these antibodies can increase immune response. In some embodiments, these antibodies can block 5T4 pathway.

The disclosure provides e.g., anti-5T4 antibodies 31G4, 32E3, 32G1, 32G8, 35B12, 35C6, the chimeric antibodies thereof, and the human or humanized antibodies thereof.

The CDR sequences for 31G4, and 31G4 derived antibodies (e.g., human or humanized antibodies) include CDRs of the heavy chain variable domain, SEQ ID NOs: 4-6, and CDRs of the light chain variable domain, SEQ ID NOs: 1-3 as defined by Kabat definition. The CDRs can also be defined by Chothia system. Under the Chothia definition, the CDR sequences of the heavy chain variable domain are set forth in SEQ ID NOs: 22-24 and CDR sequences of the light chain variable domain are set forth in SEQ ID NOs: 1-3.

Similarly, the CDR sequences for 32E3, and 32E3 derived antibodies include CDRs of the heavy chain variable domain, SEQ ID NOs: 7-9, and CDRs of the light chain variable domain, SEQ ID NOs: 1-3, as defined by Kabat definition. Under Chothia definition, the CDR sequences of the heavy chain variable domain are set forth in SEQ ID NOs: 25-27, and CDRs of the light chain variable domain are set forth in SEQ ID NOs: 1-3.

The CDR sequences for 32G1, and 32G1 derived antibodies include CDRs of the heavy chain variable domain, SEQ ID NOs: 10-12, and CDRs of the light chain variable domain, SEQ ID NOs: 1-3, as defined by Kabat definition. Under Chothia definition, the CDR sequences of the heavy chain variable domain are set forth in SEQ ID NOs: 28-30, and CDRs of the light chain variable domain are set forth in SEQ ID NOs: 1-3.

The CDR sequences for 32G8, and 32G8 derived antibodies include CDRs of the heavy chain variable domain, SEQ ID NOs: 13-15, and CDRs of the light chain variable domain, SEQ ID NOs: 1-3, as defined by Kabat definition. Under Chothia definition, the CDR sequences of the heavy chain variable domain are set forth in SEQ ID NOs: 31-33, and CDRs of the light chain variable domain are set forth in SEQ ID NOs: 1-3.

The CDR sequences for 35B12, and 35B12 derived antibodies (for example 35B12K, the VH sequence of which is set forth in SEQ ID NOs: 46) include CDRs of the heavy chain variable domain, SEQ ID NOs: 16-18, and CDRs of the light chain variable domain, SEQ ID NOs: 1-3, as defined by Kabat definition. Under Chothia definition, the CDR sequences of the heavy chain variable domain are set forth in SEQ ID NOs: 34-36, and CDRs of the light chain variable domain are set forth in SEQ ID NOs: 1-3.

The CDR sequences for 35C6, and 35C6 derived antibodies include CDRs of the heavy chain variable domain, SEQ ID NOs: 19-21, and CDRs of the light chain variable domain, SEQ ID NOs: 1-3, as defined by Kabat definition. Under Chothia definition, the CDR sequences of the heavy chain variable domain are set forth in SEQ ID NOs: 37-39, and CDRs of the light chain variable domain are set forth in SEQ ID NOs: 1-3.

The amino acid sequence for the heavy chain variable region of 31G4 antibody is set forth in SEQ ID NO: 41. The amino acid sequence for the light chain variable region of 31G4 antibody is set forth in SEQ ID NO: 40.

The amino acid sequence for the heavy chain variable region of 32E3 antibody is set forth in SEQ ID NO: 42. The amino acid sequence for the light chain variable region of 32E3 antibody is set forth in SEQ ID NO: 40.

The amino acid sequence for the heavy chain variable region of 32G1 antibody is set forth in SEQ ID NO: 43. The amino acid sequence for the light chain variable region of 32G1 antibody is set forth in SEQ ID NO: 40.

The amino acid sequence for the heavy chain variable region of 32G8 antibody is set forth in SEQ ID NO: 44. The amino acid sequence for the light chain variable region of 32G8 antibody is set forth in SEQ ID NO: 40.

The amino acid sequence for the heavy chain variable region of 35B12 antibody is set forth in SEQ ID NO: 45. The amino acid sequence for the light chain variable region of 35B12 antibody is set forth in SEQ ID NO: 40.

The amino acid sequence for the heavy chain variable region of 35C6 antibody is set forth in SEQ ID NO: 47. The amino acid sequence for the light chain variable region of 35C6 antibody is set forth in SEQ ID NO: 40.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to any one of SEQ ID NO: 41-47. In some embodiments, the light chain variable region is at least 80%, 85%, 90%, 91%, 92%, 93%, 94%, 95%, 96%, 97%, 98%, or 99% identical to SEQ ID NO: 40. The heavy chain variable region sequence can be paired with the corresponding light chain variable region sequence, and together they bind to 5T4.

Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 4-6, SEQ ID NOs: 7-9, SEQ ID NOs: 10-12, SEQ ID NOs: 13-15, SEQ ID NOs: 16-18, SEQ ID NOs: 19-21, SEQ ID NOs: 22-24, SEQ ID NOs: 25-27, SEQ ID NOs: 28-30, SEQ ID NOs: 31-33, SEQ ID NOs: 34-36, and SEQ ID NOs: 37-39; and/or one, two, or three light chain variable region CDRs selected from the group of SEQ ID NOs: 1-3.

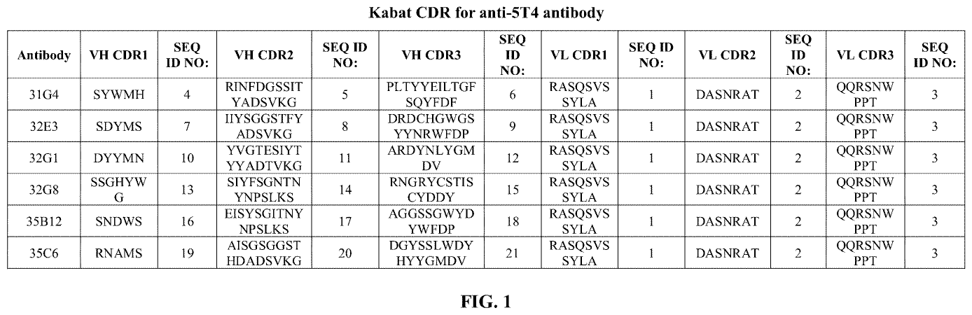

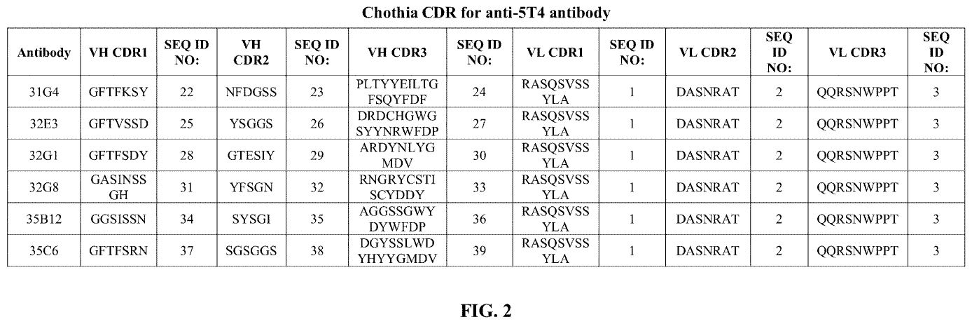

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 1 (Kabat CDR) and FIG. 2 (Chothia CDR).

In some embodiments, the antibody or an antigen-binding fragment described herein can contain a heavy chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a light chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition. In some embodiments, the CDR is determined based on Chothia definition. In some embodiments, the CDR is determined based on a combination of Kabat definition and Chothia definition.

The disclosure also provides antibodies or antigen-binding fragments thereof that bind to 5T4. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 41, and the selected VL sequence is SEQ ID NO: 40. In some embodiments, the selected VH sequence is SEQ ID NO: 42 and the selected VL sequence is SEQ ID NO: 40. In some embodiments, the selected VH sequence is SEQ ID NO: 43, and the selected VL sequence is SEQ ID NO: 40. In some embodiments, the selected VH sequence is SEQ ID NO: 44, and the selected VL sequence is SEQ ID NO: 40. In some embodiments, the selected VH sequence is SEQ ID NO: 45, and the selected VL sequence is SEQ ID NO: 40. In some embodiments, the selected VH sequence is SEQ ID NO: 46, and the selected VL sequence is SEQ ID NO: 40. In some embodiments, the selected VH sequence is SEQ ID NO: 47, and the selected VL sequence is SEQ ID NO: 40. The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

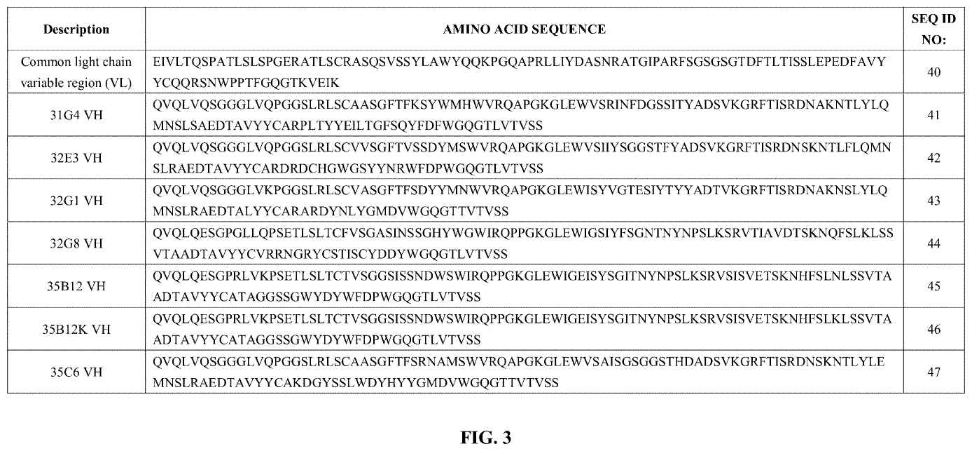

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 1 or FIG. 2, or have sequences as shown in FIG. 3. When the polypeptides are paired with corresponding polypeptide (e.g., a corresponding heavy chain variable region or a corresponding light chain variable region), the paired polypeptides bind to 5T4 (e.g., human 5T4).

The anti-5T4 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to 5T4 will retain an ability to bind to 5T4. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

One-armed antibodies can have a heavy chain and a light chain, and a heavy chain fragment comprising CH2 and CH3 domains of IgG. In some embodiments, a one-armed antibody is an antibody that only has one of the two antigen binding arms in a typical antibody. In some embodiments, a one-armed antibody comprises an antigen binding arm (e.g., VH+CH1 and VL+CL), and a Fc.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC). In some embodiments, the Fc region can be modified to enhance or increase complement-dependent cytotoxicity (CDC) or antibody-dependent cellular cytotoxicity (ADCC).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antibody or antigen-binding fragment thereof described herein recognizes an endogenous 5T4 or a recombinant 5T4. In some embodiments, the antibody or antigen-binding fragment thereof described herein recognizes human 5T4.

In some embodiments, the half-life of the antibody or antigen-binding fragment thereof described herein in wild-type mice (e.g., C57BL/6 mice) is at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the half-life of the antibody or antigen-binding fragment thereof described herein in 5T4 gene humanized mice (e.g., h5T4 mice) is at least 1 day, at least 2 days, at least 3 days, at least 4 days, or at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the max concentration (Cmax) of the antibody or antigen-binding fragment thereof described herein in 5T4 gene humanized mice (e.g., h5T4 mice) is at least 50 μg/mL, at least 75 μg/mL, at least 100 μg/mL, at least 125 μg/mL, at least 150 μg/mL, at least 175 μg/mL, or at least 200 μg/mL. In some embodiments, the serum clearance rate (CL) of the antibody or antigen-binding fragment thereof described herein in 5T4 gene humanized mice (e.g., h5T4 mice) is at least 7.5 mL/day/kg, at least 10 mL/day/kg, at least 12.5 mL/day/kg, at least 15 mL/day/kg, at least 17.5 mL/day/kg, at least 20 mL/day/kg, at least 25 mL/day/kg, at least 30 mL/day/kg, at least 35 mL/day/kg, or at least 40 mL/day/kg.

In some embodiments, the clearance rate (CL) of the antibody or antigen-binding fragment thereof described herein in wild-type mice (e.g., C57BL/6 mice) is less than 7 mL/day/kg, less than 6 mL/day/kg, less than 5 mL/day/kg, or less than 4 mL/day/kg. In some embodiments, the clearance rate (CL) of the antibody or antigen-binding fragment thereof described herein in 5T4 gene humanized mice (e.g., h5T4 mice) is less than 15 mL/day/kg, less than 14 mL/day/kg, less than 13 mL/day/kg, or less than 12 mL/day/kg.

In some embodiments, the half-life of the antibody or antigen-binding fragment thereof described herein (e.g., in FcRn gene humanized mice) is at least 6 days, at least 7 days, at least 8 days, at least 9 days, at least 10 days, at least 11 days, at least 12 days, at least 13 days, or at least 14 days. In some embodiments, the clearance rate (CL) of the antibody or antigen-binding fragment thereof described herein (e.g., in FcRn gene humanized mice) is less than 16 mL/day/kg, less than 15 mL/day/kg, less than 14 mL/day/kg, less than 13 mL/day/kg, less than 12 mL/day/kg, less than 11 mL/day/kg, less than 10 mL/day/kg, less than 9 mL/day/kg, less than 8 mL/day/kg, or less than 7 mL/day/kg.

### Antibody Drug Conjugates (ADC)

The antibodies, the antigen-binding fragments thereof, or the antigen-binding protein constructs (e.g., bispecific antibodies) described herein can be conjugated to a therapeutic agent (a drug). The therapeutic agent can be covalently or non-covalently bind to the antibody or antigen-binding fragment or the antigen binding protein construct (e.g., a bispecific antibody). In some embodiments, the bispecific antibody has a common light chain.

In some embodiments, the therapeutic agent is a cytotoxic or cytostatic agent (e.g., monomethyl auristatin E, monomethyl auristatin F, cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs). Useful classes of cytotoxic, cytostatic, or immunomodulatory agents include, for example, antitubulin agents, DNA minor groove binders, DNA replication inhibitors, and alkylating agents.

In some embodiments, the therapeutic agent can include, but not limited to, cytotoxic reagents, such as chemo-therapeutic agents, immunotherapeutic agents and the like, antiviral agents or antimicrobial agents. In some embodiments, the therapeutic agent to be conjugated can be selected from, but not limited to, MMAE (monomethyl auristatin E), MMAD (monomethyl auristatin D), or MMAF (monomethyl auristatin F).

In some embodiments, the therapeutic agent is an auristatin, such as auristatin E (also known in the art as a derivative of dolastatin-10) or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include AFP, MMAF, and MMAE. The synthesis and structure of exemplary auristatins are described in U.S. Patent Application Publication No. 2003-0083263; International Patent Publication No. WO 04/010957, International Patent Publication No. WO 02/088172, and U.S. Pat. Nos. 7,498,298, 6,884,869, 6,323,315; 6,239,104; 6,034,065; 5,780,588; 5,665,860; 5,663,149; 5,635,483; 5,599,902; 5,554,725; 5,530,097; 5,521,284; 5,504,191; 5,410,024; 5,138,036; 5,076,973; 4,986,988; 4,978,744; 4,879,278; 4,816,444; and 4,486,414, each of which is incorporated by reference herein in its entirety and for all purposes.

Auristatins have been shown to interfere with microtubule dynamics and nuclear and cellular division and have anticancer activity. Auristatins bind tubulin and can exert a cytotoxic or cytostatic effect on cancer cell. There are a number of different assays, known in the art, which can be used for determining whether an auristatin or resultant antibody-drug conjugate exerts a cytostatic or cytotoxic effect on a desired cell.

In some embodiments, the therapeutic agent is a chemotherapeutic agent. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK7; razoxane; sizofiran; spirogermanium; tenuazonic acid; triaziquone; 2′,2′,2′-trichlorotriethylamine; urethan; vindesine; dacarbazine; mannomustine; mitobronitol; mitolactol; pipobroman; gacytosine; arabinoside (“Ara-C”); cyclophosphamide; taxanes, e.g. paclitaxel (TAXOL®, Bristol-Myers Squibb Oncology, Princeton, N.J.) and doxetaxel (TAXOTERE®, Rhone-Poulenc Rorer, Antony, France); chlorambucil; gemcitabine; 6-thioguanine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4 (5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. A detailed description of the chemotherapeutic agents can be found in, e.g., US20180193477A1, which is incorporated by reference in its entirety.

In some embodiments, the antigen-binding construct is coupled to the drug via a cleavable linker e.g. a SPBD linker or a maleimidocaproyl-valine-citrulline-p-aminobenzyloxycarbonyl (VC) linker. In some embodiments, the antigen-binding construct is coupled to the drug via a non-cleavable linker e.g. a MCC linker formed using SMCC or sulfo-SMCC. Selection of an appropriate linker for a given ADC can be readily made by the skilled person having knowledge of the art and taking into account relevant factors, such as the site of attachment to the antigen binding construct, any structural constraints of the drug and the hydrophobicity of the drug (see, for example, review in Nolting, Chapter 5, Antibody-Drug Conjugates: Methods in Molecular Biology, 2013, Ducry (Ed.), Springer). A number of specific linker-toxin combinations have been described and may be used with the antigen binding constructs described herein to prepare ADCs in certain embodiments. Examples include, but are not limited to, cleavable peptide-based linkers with auristatins such as MMAE and MMAF, camptothecins such as SN-38, duocarmycins and PBD dimers; non-cleavable MC-based linkers with auristatins MMAF and MMAE; acid-labile hydrazone-based linkers with calicheamicins and doxorubicin; disulfide-based linkers with maytansinoids such as DM1 and DM4, and bis-maleimido-trioxyethylene glycol (BMPEO)-based linkers with maytansinoid DM1. Some these therapeutic agents and linkers are described, e.g., in Peters & Brown, (2015) Biosci. Rep. e00225; Dosio et al., (2014) Recent Patents on Anti-Cancer Drug Discovery 9:35-65; US Patent Publication No. US 2015/0374847, and US20180193477A1; which are incorporated herein by reference in the entirety.

Depending on the desired drug and selected linker, those skilled in the art can select suitable method for coupling them together. For example, some conventional coupling methods, such as amine coupling methods, can be used to form the desired drug-linker complex which still contains reactive groups for conjugating to the antibodies through covalent linkage. In some embodiments, a drug-maleimide complex (i.e., maleimide linking drug) can be used for the payload bearing reactive group in the present disclosure. Most common reactive group capable of bonding to thiol group in ADC preparation is maleimide. Additionally, organic bromides, iodides also are frequently used.

The ADC can be prepared by one of several routes known in the art, employing organic chemistry reactions, conditions, and reagents known to those skilled in the art (see, for example, Bioconjugate Techniques (G. T. Hermanson, 2013, Academic Press). For example, conjugation can be achieved by (1) reaction of a nucleophilic group or an electrophilic group of an antibody with a bivalent linker reagent, to form antibody-linker intermediate Ab-L, via a covalent bond, followed by reaction with an activated drug moiety D; or (2) reaction of a nucleophilic group or an electrophilic group of a drug moiety with a linker reagent, to form drug-linker intermediate D-L, via a covalent bond, followed by reaction with the nucleophilic group or an electrophilic group of an antibody. Conjugation methods (1) and (2) can be employed with a variety of antibodies, drug moieties, and linkers to prepare the ADCs described here. Various prepared linkers, linker components and toxins are commercially available or may be prepared using standard synthetic organic chemistry techniques. These methods are described e.g., in March's Advanced Organic Chemistry (Smith & March 2006, Sixth Ed., Wiley); Toki et al., (2002) J. Org. Chem. 67:1866-1872; Frisch et al., (1997) Bioconj. Chem. 7:180-186; Bioconjugate Techniques (G. T. Hermanson, 2013, Academic Press); US20210379193A1, and US20180193477A1, which are incorporated herein by reference in the entirety. In addition, a number of pre-formed drug-linkers suitable for reaction with a selected antigen binding construct are also available commercially, for example, linker-toxins comprising DM1, DM4, MMAE, MMAF or Duocarmycin SA are available from Creative BioLabs (Shirley, N.Y.).

Several specific examples of methods of preparing ADCs are known in the art and are described in U.S. Pat. No. 8,624,003 (pot method), U.S. Pat. No. 8,163,888 (one-step), and U.S. Pat. No. 5,208,020 (two-step method), and US20180193477A1, which are incorporated herein by reference in the entirety. Other methods are known in the art and include those described in Antibody-Drug Conjugates: Methods in Molecular Biology, 2013, Ducry (Ed.), Springer.

Drug loading is represented by the number of drug moieties per antibody in a molecule of ADC. For some antibody-drug conjugates, the drug loading may be limited by the number of attachment sites on the antibody. For example, where the attachment is a cysteine thiol, as in certain exemplary embodiments described herein, the drug loading may range from 0 to 8 drug moieties per antibody. In certain embodiments, higher drug loading, e.g. p≥5, may cause aggregation, insolubility, toxicity, or loss of cellular permeability of certain antibody-drug conjugates. In certain embodiments, the average drug loading for an antibody-drug conjugate ranges from 1 to about 8; from about 2 to about 6; or from about 3 to about 5. Indeed, it has been shown that for certain antibody-drug conjugates, the optimal ratio of drug moieties per antibody can be around 4. In some embodiments, the drug-to-antibody ratio (DAR) is about or at least 1, 2, 3, 4, 5, 6, 7, or 8. In some embodiments, the average DAR in the composition is about 1~ about 2, about 2~ about 3, about 3~ about 4, about 3~ about 5, about 4~ about 5, about 5~ about 6, about 6~ about 7, or about 7~ about 8.

### Antibody and ADC Characteristics

The antibodies or antigen-binding fragments thereof described herein or ADC derived therefrom can block the binding between 5T4 and 5T4 ligands.

The antibodies or antigen-binding fragments thereof as described herein or ADC derived therefrom can be 5T4 pathway agonist or antagonist. In some embodiments, by binding to 5T4, the antibody can inhibit 5T4 signaling pathway. In some embodiments, the antibody can upregulate immune response or downregulate immune response.

In some implementations, the antibody (or antigen-binding fragments thereof) or ADC derived therefrom specifically binds to 5T4 (e.g., human 5T4, monkey 5T4 (e.g., rhesus macaques, Macaca fascicularis), dog 5T4, mouse 5T4) with a dissociation rate (koff) of less than 2 s⁻¹, less than 1 s⁻¹, less than 0.1 s⁻¹, less than 0.01 s⁻¹, less than 0.001 s⁻¹, less than 0.0001 s⁻¹, less than 0.00001 s⁻¹, less than 0.000001 s⁻¹ or less than 0.0000001 s⁻¹. In some embodiments, the dissociation rate (koff) is greater than 1 s⁻¹, greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) is greater than 1×10²/Ms, greater than 1×10³/Ms, greater than 1×10⁴/Ms, greater than 1×10⁵/Ms, greater than 1×10⁶/Ms, greater than 1×10⁷/Ms or greater than 1×10⁸/Ms. In some embodiments, kinetic association rates (kon) is less than 1×10⁵/Ms, less than 1×10⁶/Ms, less than 1×10⁷/Ms, less than 1×10⁸/Ms or less than 1×10⁹/Ms.

Affinities can be deduced from the quotient of the kinetic rate constants (KD-koff/kon). In some embodiments, KD is less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., ELISA, RIA, and surface plasmon resonance (SPR). In some embodiments, the antibody binds to human 5T4, monkey 5T4, dog 5T4, and/or mouse 5T4. In some embodiments, the antibody does not bind to human 5T4, monkey 5T4, dog 5T4, and/or mouse 5T4.

In some embodiments, the antibodies or antigen-binding fragments thereof as described herein can bind to 5T4 expressing cells with a half maximal effective concentration (EC50) that is lower than 3.5 ug/mL, lower than 3 μg/mL, lower than 2.5 μg/mL, lower than 2 μg/mL, lower than 1.5 μg/mL, lower than 1 μg/mL, lower than 0.75 μg/mL, lower than 0.5 μg/mL, lower than 0.25 μg/mL, or lower than 0.15 ug/mL.

In some embodiments, the antibodies or antigen-binding fragments thereof as described herein can bind to 5T4 expressing cells. In some embodiment, the percentage of positive cells is above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%.

In some embodiments, the antibody or antigen-binding fragment thereof as described herein or ADC derived therefrom is added to NCI-H2030 cells (ATCC, Cat #: CRL-5914), CMT cells (ATCC, Cat #: CRL-3456) or D17 cells (ATCC, Cat #: CCL-183) to test the endocytosis rate. In some embodiments, the antibody or antigen-binding fragment thereof as described herein or ADC derived therefrom has an endocytosis rate of above 5%, above 10%, above 15%, above 20%, above 25%, above 30%, above 35%, above 40%, above 45%, above 50%, above 55%, above 60%, above 65%, above 70%, above 75%, above 80%, above 85%, above 90%, above 91%, above 92%, above 93%, above 94%, above 95%, above 96%, above 97%, or above 98%.

In some embodiments, thermal stabilities are determined. The antibodies or antigen binding fragments as described herein or ADC derived therefrom can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen-binding fragments thereof as described herein or ADC derived therefrom can bind to the same epitope of 5T4. In some embodiments, the antibodies or antigen-binding fragments thereof as described herein or ADC derived therefrom can bind to different epitopes of 5T4.

In some embodiments, the antibodies or antigen-binding fragments thereof as described herein have a purity of above 80%, above 85%, above 90%, above 91%, above 92%, above 93%, above 94%, above 95%, above 96%, above 97%, or above 98%, as determined by size exclusion chromatography (SEC). In some embodiments, the antibodies or antigen-binding fragments thereof as described herein have a hydrophobic interaction chromatography (HIC) retention time that is higher than 2 minutes, higher than 3 minutes, higher than 4 minutes, higher than 5 minutes, higher than 6 minutes, higher than 7 minutes, higher than 8 minutes, higher than 9 minutes, higher than 10 minutes, higher than 11 minutes, higher than 12 minutes, higher than 13 minutes, higher than 14 minutes, higher than 15 minutes, higher than 16 minutes, higher than 17 minutes, higher than 18 minutes, higher than 19 minutes, higher than 20 minutes, higher than 21 minutes or higher than 22 minutes.

In some embodiments, the antibodies or antigen-binding fragments thereof as described herein have a main peak that constitutes more than 50%, more than 55%, more than 60%, more than 65%, more than 70%, more than 75%, more than 80%, more than 85%, or more than 90%, as determined by capillary isoelectric focusing (cIEF). In some embodiments, the antibodies or antigen-binding fragments thereof as described herein have an acidic peak that constitutes more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, or more than 90%, as determined by capillary isoelectric focusing (cIEF). In some embodiments, the antibodies or antigen-binding fragments thereof as described herein have a isoelectric point (PI) that is higher than 2, higher than 2.5, higher than 3, higher than 3.5, higher than 4, higher than 4.5, higher than 5, higher than 5.5, higher than 6, higher than 6.5, higher than 7, higher than 7.5 higher than 8, higher than 8.5, higher than 9, higher than 9.5, higher than 10, higher than 10.5, higher than 11, higher than 11.5, higher than 12, higher than 12.5, higher than 13, higher than 13.5, higher than 14, higher than 14.5, higher than 15, higher than 15.5, higher than 16, higher than 16.5, higher than 17, higher than 17.5, higher than 18, or higher than 18.5, as determined by capillary isoelectric focusing (cIEF). In some embodiments, the antibodies or antigen-binding fragments thereof as described herein have a half maximal effective concentration (EC50) that is lower than 2.5, lower than 2, lower than 1.5, lower than 1, lower than 0.75, lower than 0. 5, lower than 0.25, lower than 0.15 μg/mL.

In some embodiments, the ADC described herein has an average drug-to-antibody ratio (DAR) of higher than 3, higher than 3.2, higher than 3.4, higher than 3.6, higher than 3.8, higher than 4, higher than 4.2, higher than 4.4, or higher than 4.6, as determined by HPLC. In some embodiments, the ADC described herein has an average DAR of lower than 3, lower than 3.2, lower than 3.4, lower than 3.6, lower than 3.8, lower than 4, lower than 4.2, lower than 4.4 or lower than 4.6, as determined by HPLC.

In some embodiments, the antibody or antigen-binding fragment thereof as described herein or ADC derived therefrom has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody or antigen-binding fragment thereof as described herein or ADC derived therefrom has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38 or 39 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

TGI (%)=[1−(Ti−T0)/(Vi−V0)]×100%

Ti is the average tumor volume in the treatment group on day i. T0 is the average tumor volume in the treatment group on day zero. Vi is the average tumor volume in the control group on day i. V0 is the average tumor volume in the control group on day zero.

In some embodiments, the antibodies or antigen-binding fragments thereof as described herein or ADC derived therefrom are 5T4 antagonists. In some embodiments, the antibodies or antigen binding fragments as described herein or ADC derived therefrom decrease 5T4 signal transduction in a target cell that expresses 5T4.

In some embodiments, the antibodies or antigen binding fragments as described herein or ADC derived therefrom can enhance APC (e.g., DC cell) function, for example, inducing surface expression of costimulatory and MHC molecules, inducing production of proinflammatory cytokines, and/or enhancing T cell triggering function.

In some embodiments, the antibodies or antigen binding fragments as described herein or ADC derived therefrom can bind to tumor cells that express 5T4. In some embodiments, the antibodies or antigen binding fragments as described herein or ADC derived therefrom can induce complement-dependent cytotoxicity (CDC) and/or antibody dependent cellular cytoxicity (ADCC), and kill the tumor cell.

In some embodiments, the antibodies or antigen binding fragments as described herein or ADC derived therefrom have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments as described herein or ADC derived therefrom can induce complement complement-dependent cytotoxicity (CDC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments as described herein or ADC derived therefrom do not have a functional Fc region. For example, the antibodies or antigen binding fragments are Fab, Fab′, F(ab′)₂, and Fv fragments. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and I332E mutations according to EU numbering). In some embodiments, the Fc has N297A mutation according to EU numbering. In some embodiments, the Fc has YTE mutations (M252Y, S254T and T256E according to EU numbering).

### Methods of Making Anti-5T4 Antibodies

An isolated fragment of human 5T4 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of 5T4 and encompasses an epitope of the protein such that an antibody raised against the peptide forms a specific immune complex with the protein. As described above, the full length sequence of human 5T4 is known in the art. In some embodiments, an Fc-tagged or His-tagged human 5T4 protein is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human 5T4). The preparation can further include an adjuvant, such as Freund's complete or incomplete adjuvant, or a similar immunostimulatory agent.

Polyclonal antibodies can be prepared as described above by immunizing a suitable subject with a 5T4 polypeptide, or an antigenic peptide thereof (e.g., part of 5T4) as an immunogen. The antibody titer in the immunized subject can be monitored over time by standard techniques, such as with an enzyme-linked immunosorbent assay (ELISA) using the immobilized 5T4 polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., 5T4. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-5T4 antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chain. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant region (e.g., IgG1, IgG2, IgG3, and IgG4).

Identity or homology with respect to an original sequence is usually the percentage of amino acid residues present within the candidate sequence that are identical with a sequence present within the human, humanized, or chimeric anti-5T4 antibody or fragment, after aligning the sequences and introducing gaps, if necessary, to achieve the maximum percent sequence identity, and not considering any conservative substitutions as part of the sequence identity.

Additional modifications to the anti-5T4 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-5T4 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues; or position 314 in Kabat numbering); however, Asn297 may also be located about ±3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, genitourinary cancer or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors. In some embodiments, the cancer is colorectal cancer, gastric cancer, ovarian cancer, lung cancer, head and neck cancer, or pancreatic cancer.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder. These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-5T4 antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has dermatological disorders, liver disease (e.g., cirrhosis), Hidradenitis, experimental autoimmune encephalomyelitis. In some embodiments, the subject has renal disease, lupus, Sjogren's syndrome, ulcerative colitics, psoriasis, Hidradenitis suppurativa, Immune Thrombocytopenia (ITP), or other inflammatory arthritis. In some embodiments, the subject has multiple sclerosis or myasthenia gravis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus. In some embodiments, the methods involve administering to the subject an effective amount of a composition comprising the antibody or antigen-binding fragment thereof as described herein.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of OX40, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, a HER2 agonist and a OX40 agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, anti-ICOS antibody, anti-CD27 antibody, anti-OX40 antibody, anti-4-1BB antibody, anti-CD40 antibody, and/or an anti-GITR antibody.

In one aspect, the disclosure provides a combination therapy. In some embodiments, the anti-5T4 antibody or antigen-binding fragment thereof (e.g., any antibody described herein) can be administered together with an anti-OX40 antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50: ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 g/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### Examples

The invention is further described in the following examples, which do not limit the scope of the invention described in the claims.

### Example 1. Generating Anti-5T4 Antibody

His-tagged human 5T4 protein (h5T4-His, Sino Biological, Cat #: 19845-H08H) and mouse 5T4 protein (m5T4-His, Novoprotein, Cat #: C00M) were emulsified with adjuvants, and was used to immunize RenLite™ mice (Biocytogen, complete human heavy chain variable domain combined with a common light chain substitution in situ). The RenLite™ mice are described, e.g., in PCT/CN2021/097652, which is incorporated herein by reference in its entirety. Before immunization, retro-orbital blood was collected as a negative control.

Freund's complete adjuvant (CFA) was used for the first immunization and Freund's incomplete adjuvant (IFA) was used for the second, third, and fourth immunizations. A total of four immunizations were performed. The first and second immunizations were separated by two weeks, and the remaining immunizations were separated by one week. One week after the fourth immunization, retro-orbital blood was collected, and the antibody titer of serum was detected by fluorescence activated cell sorting (FACS). For impulse immunization, one week later mice with high titer were further injected with h5T4-His protein and m5T4-His protein through intraperitoneal injection, and CHO-S cell expressing human 5T4 through the tail vein injection, respectively.

Antigen-specific immune cells were isolated from the immunized mice to further obtain anti-5T4 antibodies or to obtain the light chain and heavy chain variable region sequences of the anti-5T4 antibodies. For example, single cell technology (e.g., Beacon® Optofluidic System, Berkeley Lights Inc.) was used to screen and find plasma cells secreting antigen-specific monoclonal antibodies, and then reverse transcription and PCR sequencing were used to obtain antibody variable region sequences. The obtained variable region sequences were cloned into a vector containing a sequence encoding the human IgG1 constant region for antibody expression. Binding of the expressed antibodies to 5T4 was verified by FACS. Exemplary antibodies obtained by this method included: 31G4, 32E3, 32G1, 32G8, 35B12 and 35C6.

These antibodies contained substantially the same light chain, and their VH CDR1, CDR2, CDR3 and VL CDR1, CDR2, CDR3 sequences are shown in FIG. 1 or FIG. 2. The VH and VL regions of the antibodies and derived antibody are shown in FIG. 3.

### Example 2. Cross-Species Binding of Anti-5T4 Antibodies

CHO-h5T4 cells, CHO-m5T4 cells, CHO-fas5T4 cells or CHO-d5T4 cells were transferred to a 96-well plate at a density of 2×10⁵ cells/well respectively. The anti-5T4 antibodies were added to the 96-well plate, and incubated at 4° C. for 30 min. Then, the cells were incubated with the secondary antibody anti-hlgG-Fc-Alex Flour 647 (RL1-H) (Jackson ImmunoResearch Laboratories, Inc., Cat #: 109-606-170) at 4° C. in the dark for 15 minutes before flow cytometry analysis. The test results are shown in the table below.

CHO-h5T4 cells, CHO-m5T4 cells, CHO-fas5T4 cells and CHO-d5T4 cells were obtained by transfecting CHO-S cells with vectors expressing human 5T4 (SEQ ID NO: 48), mouse 5T4 (SEQ ID NO: 49), monkey (Macaca fascicularis) 5T4 (SEQ ID NO: 50) and dog 5T4 (SEQ ID NO: 51), respectively.

TABLE 1

| | | Percentage | |
|---|---|---|---|
| | | of Positive | |
| Antibody | CHO cells | Cells | Evaluation |
| NC | CHO-h5T4 | 1.0% | No Binding |
| | CHO-m5T4 | 0.7% | No Binding |
| | CHO-fas5T4 | 1.3% | No Binding |
| | CHO-d5T4 | 1.0% | No Binding |
| NC + secondary | CHO-h5T4 | 2.0% | No Binding |
| antibody | CHO-m5T4 | 0.2% | No Binding |
| | CHO-fas5T4 | 1.6% | No Binding |
| | CHO-d5T4 | 0.2% | No Binding |
| 31G4 | CHO-h5T4 | 79.3% | Binding |
| | CHO-m5T4 | 1.4% | No binding |
| | CHO-fas5T4 | 58.8% | Binding |
| | CHO-d5T4 | 1.1% | No binding |
| 32E3 | CHO-h5T4 | 65.8% | Binding |
| | CHO-m5T4 | 52.2% | Binding |
| | CHO-fas5T4 | 51.5% | Binding |
| | CHO-d5T4 | 53.7% | Binding |
| 32G1 | CHO-h5T4 | 56.5% | Binding |
| | CHO-m5T4 | 27.1% | Binding |
| | CHO-fas5T4 | 53.2% | Binding |
| | CHO-d5T4 | 56.3% | Binding |
| 32G8 | CHO-h5T4 | 61.5% | Binding |
| | CHO-m5T4 | 0.7% | No binding |
| | CHO-fas5T4 | 52.6% | Binding |
| | CHO-d5T4 | 62.8% | Binding |
| 35B12 | CHO-h5T4 | 78.0% | Binding |
| | CHO-m5T4 | 54.1% | Binding |
| | CHO-fas5T4 | 52.1% | Binding |
| | CHO-d5T4 | 76.0% | Binding |
| 35C6 | CHO-h5T4 | 67.3% | Binding |
| | CHO-m5T4 | 63.3% | Binding |
| | CHO-fas5T4 | 55.7% | Binding |
| | CHO-d5T4 | 71.1% | Binding |

All anti-5T4 antibodies can bind to human 5T4, monkey 5T4 and dog 5T4 (except 31G4). In addition, 32E3, 32G1, 35B12 and 35C6 can also bind to mouse 5T4.

### Example 3. Internalization of Anti-5T4 Antibodies

Anti-5T4 antibodies (2.5 μg/mL) and pHAb-AffiniPure Fab Goat Anti-Human IgG Secondary Antibody were added to the NCI-H2030 cells (ATCC, Cat #: CRL-5914), CMT cells (ATCC, Cat #: CRL-3456) or D17 cells (ATCC, Cat #: CCL-183) and incubated for 6-24 hours. After incubating, the cells were centrifuged and washed in FACS buffer. MFI was detected on a flow cytometer, and the endocytosis rates of anti-5T4 antibodies were calculated. The results are summarized in the table below.

PF06263507 is a humanized IgG1 monoclonal antibody targeting 5T4, and the VH and VL sequences are shown in SEQ ID NOs: 52-53. Human IgG1 protein was used as isotype control (ISO).

TABLE 2

| | NCI-H2030 | | CMT | | D17 | |
|---|---|---|---|---|---|---|
| | | Positive | | Positive | | Positive |
| Antibody | MFI | cells | MFI | cells | MFI | cells |
| ISO | 12996 | 1.4% | 21559 | 2.5% | 21347 | 1.5% |
| PF06263507 analog | 55695 | 88.5% | 30422 | 15.5% | 30156 | 9.0% |
| 31G4 | 22494 | 7.7% | 20268 | 1.5% | 20089 | 1.0% |
| 32E3 | 79341 | 93.6% | 19981 | 1.9% | 19880 | 1.1% |
| 32G1 | 40854 | 69.3% | 22394 | 2.4% | 22213 | 1.2% |
| 32G8 | 32585 | 44.4% | 29708 | 14.2% | 29470 | 8.4% |
| 35B12 | 41806 | 69.5% | 23372 | 5.1% | 23212 | 2.8% |
| 35C6 | 48102 | 80.5% | 40647 | 38.7% | 40598 | 30.2% |

The data showed that, 31G4, 32E3, 32G1, 32G8, 35B12 and 35C6 exhibited good endocytosis rates in NCI-H2030 cells, CMT cells and D17 cells.

### Example 4. Binding Affinity of Anti-5T4 Antibodies

The binding affinity of the anti-5T4 antibodies to His-tagged 5T4 protein of human (h5T4, ACRO Biosystems Inc., Cat #: TPG-H52E5), monkey (fas5T4, ACRO Biosystems Inc., Cat #: TPG-C52H3) or mouse (m5T4, ACRO Biosystems Inc., Cat #: TPG-M52H3) was verified using Biacore™ (Biacore, Inc., Piscataway N.J.) 8K biosensor equipped with pre-immobilized Protein A sensor chips.

Purified anti-5T4 antibodies were diluted to 2 μg/mL and then injected into the Biacore™ 8K biosensor at 10 μL/min for about 50 seconds to achieve a desired protein density (e.g., about 50 response units (RU)). His-tagged 5T4 protein at a concentration of 200 nM was then injected at 30 μL/min for 180 seconds. Dissociation was monitored for 400 seconds. The chip was regenerated after the last injection of each titration with Glycine (pH 2.0), 30 μL/min for 30 seconds. For ISO control, an antibody targeting an irrelevant target protein was used.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD-koff/kon).

As a person of ordinary skill in the art would understand, the same method with appropriate adjustments for parameters (e.g., antibody concentration) was performed for each tested antibody. The results for the tested antibodies are summarized in the table below.

TABLE 3

| | 5T4 | kon | koff | KD |
|---|---|---|---|---|
| Antibody | protein | (1/Ms) | (1/s) | (M) |
| ISO | h5T4 | — | — | No binding |
| | fas5T4 | — | — | No binding |
| | m5T4 | — | — | No binding |
| MAB2 | h5T4 | 1.30E+05 | 2.92E−03 | 2.25E−08 |
| analog-SI | fas5T4 | 9.90E+04 | 5.45E−03 | 5.51E−08 |
| | m5T4 | — | — | No binding |
| 31G4 | h5T4 | 1.48E+05 | 3.97E−04 | 2.68E−09 |
| | fas5T4 | 1.00E+05 | 1.01E−03 | 1.01E−08 |
| | m5T4 | — | — | No binding |
| 32E3 | h5T4 | 4.49E+04 | 6.61E−03 | 1.47E−07 |
| | fas5T4 | 2.56E+04 | 2.97E−03 | 1.16E−07 |
| | m5T4 | — | — | No binding |
| 32G1 | h5T4 | 6.77E+04 | 2.49E−02 | 3.68E−07 |
| | fas5T4 | 1.75E+08 | 1.23E+01 | 7.01E−08 |
| | m5T4 | — | — | No binding |
| 32G8 | h5T4 | 1.24E+05 | 5.29E−03 | 4.27E−08 |
| | fas5T4 | 9.80E+04 | 1.43E−02 | 1.46E−07 |
| | m5T4 | — | — | No binding |
| 35B12 | h5T4 | 1.76E+05 | 2.15E−02 | 1.22E−07 |
| | fas5T4 | 1.11E+05 | 2.19E−02 | 1.98E−07 |
| | m5T4 | 5.07E+04 | 3.66E−03 | 7.22E−08 |
| 35C6 | h5T4 | 1.64E+05 | 3.15E−03 | 1.92E−08 |
| | fas5T4 | — | — | Weak binding |
| | m5T4 | 3.10E+05 | 4.07E−04 | 1.31E−09 |

MAB2 is a humanized IgG1 monoclonal antibody targeting 5T4, and the VH and VL sequences are shown in SEQ ID NOs: 54-55. Human IgG1 protein was used as isotype control (ISO).

The results show that the 31G4, 32E3, 32G1, 32G8, 35B12 and 35C6 can all bind to human 5T4 and monkey 5T4. In addition, 35B12 and 35C6 also bind to mouse 5T4 with high affinity.

### Example 5. Epitope Assays of Anti-5T4 Antibodies

Epitope binding assays were performed using ForteBio Octet system to determine whether two anti-5T4 antibodies target the same or overlapping epitopes. 1×HBS-EP+buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH7.4) diluted from HBS-EP+buffer (10×) was used as the running buffer throughout the experiment. About 50 RU of h5T4-His protein was captured at a flow rate of 10 μL/min, and 200 nM of the tested antibody was injected at a flow rate of 30 μL/min to bind the ligand. Another comparison antibody was injected under the same conditions to determine whether the binding of different antibodies interfered with each other. The binding time was 180 s for each antibody.

The binding value of each antibody was obtained using Data Analysis HT 12.0. To quantify the interference of one antibody binding to another, a binding ratio was calculated to compare each pair of antibodies. The binding ratio is defined as the binding value of the second antibody (analyte 2), divided by the binding value of the first antibody (analyte 1). The binding ratio of each antibody pair was summarized in the table below. More specifically, the binding ratio was between-0.02 to 0.5, if analyte 1 exhibited a blocking effect to analyte 2. The binding ratio was between 0.5-1.05, if analyte 1 did not exhibit a blocking effect to analyte 2. In general, antibody pairs that interfere with each other have the same or overlapping epitopes.

The results showed that 31G4, 32E3, 32G8 and 32G1 recognize different epitopes. 35B12 and 35C6 target the same or overlapping epitopes.

TABLE 4

| | Analyte 1 | | | | | |
|---|---|---|---|---|---|---|
| Analyte 2 | 31G4 | 32E3 | 32G1 | 32G8 | 35B12 | 35C6 |
| 31G4 | 0.04 | 1.01 | 1.04 | 1.01 | 1.01 | 1.05 |
| 32E3 | 0.80 | −0.01 | 0.83 | −0.06 | 0.97 | 0.96 |
| 32G1 | 0.89 | 0.89 | 0.01 | 0.82 | −0.02 | 1.03 |
| 32G8 | 0.85 | 0.04 | 1.01 | 0.11 | 0.96 | 1.01 |
| 35B12 | 0.87 | 0.97 | 0.05 | 0.91 | 0.00 | 0.04 |
| 35C6 | 0.81 | 0.93 | 0.96 | 0.98 | 0.03 | 0.02 |

### Example 6. Stability Analysis of Anti-5T4 Antibodies

The biophysical properties and stability of the anti-5T4 antibodies were evaluated. Antibody sample processing procedures are as follows:

1) The antibody was placed in a buffer solution containing 3 mg/ml histidine, 80 mg/ml sucrose, and 0.2 mg/ml Tween 80 at pH 6.0, to reach a final concentration of 2 mg/mL, and kept in sealed Eppendorf tubes at 40±2° C., 60%±5% RH (hereinafter referred to as 40° C.) for 7 days, and the thermal stability was evaluated.

2) The antibody was loaded into protein A column and eluted with a buffer (0.1 mol/L HAc) at pH 3.5. Half of the antibody was added into 2M Tris buffer to adjust the pH to 7.5 immediately. The remaining half was kept at pH 3.5 for 6 hours before adjusting the pH to 7.5. The diluted antibody was kept in sealed Eppendorf tubes at pH 3.5±0.1, 25±2° C. (hereinafter referred to as pH 3.5) for 6 hours to test stability at low pH.

The following tests were performed: (1) detecting the purity of antibodies by Size-Exclusion High Performance Liquid Chromatography (SEC-HPLC) (indicated as the percentage of the main peak area to the sum of all peak areas (Purity, %)); (2) detecting the apparent hydrophobicity of the antibodies using the Hydrophobic Interaction Chromatography-High Performance Liquid Chromatography (HIC-HPLC) method (indicated as the retention time of the main peak (HIC, min)); (3) detecting the pI (isoelectric point) and charge variants of the antibodies by the Capillary Isoelectric Focusing (cIEF) method (indicated as the percentages of the main component, acidic component, and alkaline component).

In the SEC-HPLC experiments, the antibody samples were diluted to 1 mg/mL with purified water and an Agilent 1290 chromatograph system (connected with XBridge Protein BEH SEC column (200 Å, Waters Corporation)) was used. The following parameters were used: mobile phase: 0.1Mphosphate buffer (PB)+10% ACN, pH7.4; flow rate: 1.8 ml/min; column temperature: 25° C.; detection wavelength: 280 nm, 220 nm; injection volume: 10 μL; sample tray temperature: about 4° C.; and running time: 7 minutes.

In the HIC-HPLC experiments, an Agilent 1260 chromatograph system (connected with ProPac HIC-10 column (4.6×250 mm, Thermo Scientific)) was used, and samples were 10 times diluted using mobile phase A. The following parameters were used: mobile phase A: 0.9 M ammonium sulfate, 0.1M phosphate buffer (PB), 10% acetonitrile pH 6.5; mobile phase B: 0.1M phosphate buffer (PB), 10% acetonitrile pH 6.5; flow rate: 0.8 ml/min; gradient: 0 min 100% A, 2 min 100% A, 32 min 100% B, 34 min 100% B, 35 min 100% A, and 45 min 100% A; column temperature: 30° C.; detection wavelength: 280 nm,220 nm; injection volume: 10 μg; sample tray temperature: about 10° C.; and running time: 50 minutes.

In the cIEF experiments, a Maurice cIEF Method Development Kit (Protein Simple, Cat #: PS-MDK01-C) was used for sample preparation. Specifically, 8 μL, 30 ug protein sample was mixed with the following reagents in the kit: 1 μL Maurice cIEF pI Marker-7.05, 1 μL Maurice cIEF pI Marker-10.10, 35 μL 1% Methyl Cellulose Solution, 2 μL Maurice cIEF 500 mM Arginine, 1.33 μL Ampholytes (Pharmalyte pH ranges 3-10), and 6.66 μL Ampholytes (Pharmalyte pH ranges 8-10.5) water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes.

Detailed results are shown in the table below. The results showed that these anti-5T4 antibodies have good stability and physical and chemical properties.

TABLE 5

| | | | | cIEF | | |
|---|---|---|---|---|---|---|
| | | SEC | | Acidic | Main | |
| | | Purity | HIC | peak | peak | Alkali |
| Antibody | Treatment | (%) | (min) | (%) | (%) | peak |
| 31G4 | 0 d | 82.01 | 13.701 | 30.4 | 62.5 | 7.1 |
| | 40° C. 7 d | 86.63 | 13.746 | 33.8 | 59.1 | 7.1 |
| | pH3.5 0 h | 88.76 | 13.739 | 33.2 | 60.5 | 6.3 |
| | pH3.5 6 h | 90.55 | 13.725 | 31.1 | 62.9 | 6.0 |
| 32E3 | 0 d | 92.08 | 16.224 | 23.3 | 59.5 | 17.2 |
| | 40° C. 7 d | 92.14 | 16.225 | 26.6 | 55.4 | 18.0 |
| | pH3.5 0 h | 94.45 | 16.257 | 23.9 | 60.2 | 15.9 |
| | pH3.5 6 h | 95.92 | 16.243 | 23.4 | 59.8 | 16.8 |
| 32G1 | 0 d | 96.68 | 4.560 | 28.5 | 64.4 | 7.0 |
| | 40° C. 7 d | 96.76 | 4.552 | 34.1 | 58.0 | 7.9 |
| | pH3.5 0 h | 94.56 | 2.173 | 22.9 | 70.7 | 6.5 |
| | pH3.5 6 h | 94.35 | 2.173 | 23.0 | 71.3 | 5.7 |
| 32G8 | 0 d | 91.50 | 2.521 | 7.8 | 86.7 | 5.5 |
| | 40° C. 7 d | 92.72 | 2.520 | 10.9 | 86.3 | 2.8 |
| | pH3.5 0 h | 97.01 | 2.560 | 6.2 | 89.0 | 4.8 |
| | pH3.5 6 h | 96.99 | 2.541 | 5.7 | 89.5 | 4.9 |
| 35B12 | 0 d | 94.19 | 21.344 | 12.9 | 83.5 | 3.6 |
| | 40° C. 7 d | 95.89 | 21.321 | 18.0 | 77.0 | 4.5 |
| | pH3.5 0 h | 97.87 | 21.392 | 13.1 | 83.4 | 3.5 |
| | pH3.5 6 h | 97.68 | 21.389 | 13.3 | 83.2 | 3.5 |
| 35C6 | 0 d | 95.98 | 7.739 | 14.6 | 81.4 | 4.0 |
| | 40° C. 7 d | 96.05 | 7.741 | 21.7 | 73.4 | 4.9 |
| | pH3.5 0 h | 100 | 7.690 | 14.6 | 80.9 | 4.5 |
| | pH3.5 6 h | 98.74 | 7.693 | 15.1 | 80.4 | 4.5 |

### Example 7. Generating Anti-5T4 Antibody Drug Conjugates (ADC)

Each purified antibody (31G4, 32E3, 32G1, 32G8, 35B12 and 35C6) was coupled with MMAE (monomethyl auristatin E) or MMAF (monomethyl auristatin F) through a maleimidocaproyl-valine-citrulline-p-aminobenzyloxycarbonyl (VC) linker.

For the names of antibody drug conjugates, “ADC” is added directly after the antibody name. For example, if 31G4 is coupled to MMAE, it is named as 31G4-ADC.

HIC-HPLC was performed to detect the coupling of antibodies with drug molecules. The results show that the drug-to-antibody ratio (DAR) of ADC is about 4.

### Example 8. Anti-Tumor Activity of Anti-5T4 ADC in NUGC-4 Model

B-NDG mice (Biocytogen Pharmaceuticals (Beijing) Co., Ltd., Cat #: B-CM-002) were used to determine the anti-tumor activity of anti-5T4 ADC. About 5×10⁶ stomach cancer NUGC-4 cells were injected subcutaneously in B-NDG mice, and when the tumor volume grew to about 200 mm3, the mice were divided to a control group and two treatment groups based on tumor size (5 mice per group). The treatment groups were randomly injected with 3 mg/kg 32G1-ADC (G2) or 3 mg/kg PF06263507-ADC (G3) by intravenous (i.v.) injection. The control group mice were injected with phosphate buffer saline (PBS) (G1). The frequency of administration was once a week (2 administrations in total). PF06263507 analog was coupled with MMAE as a positive control (named PF06263507-ADC). The tumor volume was measured twice a week and the body weight of the mice was weighed as well. Euthanasia was performed when the tumor volume of the mouse reached 3000 mm3.

The length of the long axis and the short axis of the tumor were measured and the volume of the tumor was calculated as 0.5×(long axis)×(short axis)². The tumor growth inhibition percentage (TGI %) was calculated using the following formula: TGI (%)=[1−(Ti−T0)/(Vi−V0)]×100%. Ti is the average tumor volume in the treatment group on day i. TO is the average tumor volume in the treatment group on day zero. Vi is the average tumor volume in the control group on day i. VO is the average tumor volume in the control group on day zero. T-test was performed for statistical analysis. A TGI % higher than 60% indicates clear suppression of tumor growth. P<0.05 is a threshold to indicate significant difference.

At the end of the experiment, there was no significant difference between each group, and all mice in treatment groups survived at the end of the experiment, which showed that 32G01-ADC was well tolerated and not obviously toxic to the mice.

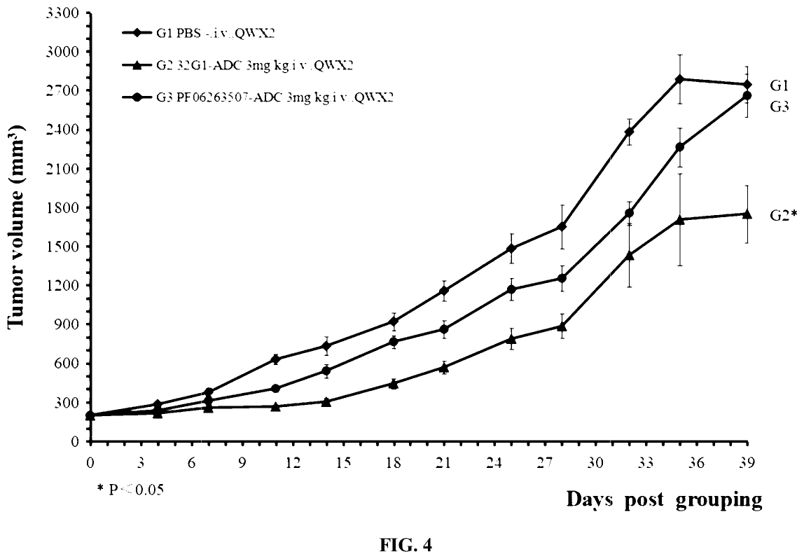

The tumor size data in groups treated with the ADCs are shown in FIG. 4. The table below summarizes the results for this experiment, including the tumor volumes on the day of grouping (Day 0), 14 days after grouping (Day 14), 28 days after grouping (Day 28) and at the end of the experiment (Day 39); the survival rate of the mice on Day 39; Tumor Growth Inhibition value (TGI); and the statistical differences (P value) of tumor volume between the treatment and control groups.

TABLE 6

| | Tumor volume (mm³) | | | | | TGI % | TGI % | P value |
|---|---|---|---|---|---|---|---|---|
| Group | Day 0 | Day 14 | Day 28 | Day 39 | Survival | (Day 28) | (Day 39) | (Day 39) |
| G1 | 198 ± 4 | 732 ± 72 | 1651 ± 168 | 2745 ± 138 | 3/5 | NA | NA | NA |
| G2 | 199 ± 6 | 303 ± 29 | 884 ± 95 | 1750 ± 220 | 5/5 | 52.8% | 39.1% | 0.017 |
| G3 | 199 ± 9 | 541 ± 54 | 1253 ± 98 | 2661 ± 165 | 5/5 | 27.4% | 3.3% | 0.738 |

As shown in FIG. 4 and the table above, compared with the control group, the tumor growth in the treatment groups were inhibited to different extents. 32G1-ADC treatment group exhibited better tumor inhibitory effect as compared to that of the positive control PF06263507-ADC. Particularly, at the end of the experiment (Day 39), 32G1-ADC still showed stronger anti-tumor effect with a TGI % of 39.1% than that of the positive control (with a TGI % of 3.3%).

### Example 9. Anti-Tumor Activity in Lung Cancer Patient-Derived Xenograft Model

The ADCs were tested for their effects on tumor growth in vivo in a model of lung cancer patient-derived xenograft model. Immunofluorescence staining of patient-derived tumor fragments was performed and the images were analyzed via a HALO® image analysis platform (3.2 version). The results showed that 5T4 positive cells constituted 58.86% of total cells in the human lung tumor tissues. Specifically, the patient-derived tumor fragments (2 mm×2 mm×2 mm) were engrafted in the right flank of B-NDG mice. When the tumors in the mice reached a volume of about 250-300 mm3, the mice were randomly placed into different groups based on tumor volumes. The mice were then injected with PBS or ADCs. Details are shown in the table below.

TABLE 7

| | No. | | | | | |
|---|---|---|---|---|---|---|
| | of | | | | Fre- | Total No. of |
| Group | mice | Antibodies | Dosage | Route | quency | administration |
| G1 | 5 | PBS | — | i.v. | QW | 2 |
| G2 | 5 | ISO-ADC | 3 mg/kg | i.v. | QW | 2 |
| G3 | 5 | 32G1-ADC | 3 mg/kg | i.v. | QW | 2 |
| G4 | 5 | PF06263507-ADC | 3 mg/kg | i.v. | QW | 2 |

The body weights were measured twice a week. During the experiment, there was no significant difference in body weights among groups, indicating that the tested ADCs were well tolerated and were not obviously toxic to the mice.

The table below summarizes the results for this experiment, including the tumor volumes on the day of grouping (Day 0), 14 days after grouping (Day 14) and 28 days after grouping (Day 28); TGI (%); and the statistical differences (P value) of tumor volumes and body weights between the treatment and control groups.

TABLE 8

| | | | | | | P value (Day 28) | |
|---|---|---|---|---|---|---|---|
| | Tumor volume (mm³) | | | Survival | TGI (%) | Tumor | Body |
| Group | Day 0 | Day 14 | Day 28 | (Day 28) | (Day 28) | volume | weight |
| G1 | 250 ± 14 | 824 ± 63 | 1736 ± 197 | 5/5 | NA | NA | NA |
| G2 | 249 ± 31 | 545 ± 98 | 1826 ± 303 | 5/5 | −6.1 | 0.810 | 0.070 |
| G3 | 250 ± 28 | 100 ± 43 | 259 ± 107 | 5/5 | 99.3 | 1.70E−04 | 0.574 |
| G4 | 251 ± 25 | 192 ± 16 | 486 ± 39 | 5/5 | 84.2 | 2.49E−04 | 0.062 |

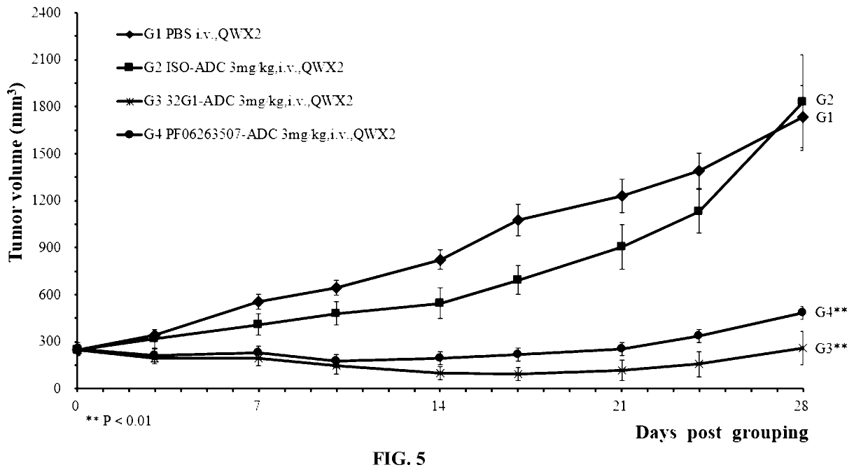

The tumor volumes of mice in different groups are shown in FIG. 5. The treatment groups (G3-G4) showed better tumor inhibition effects compared with PBS group (G1). In addition, 32G1-ADC (G3) showed a higher TGI % on Day 28 than that of the positive control PF06263507-ADC (G4) and ISO-ADC (G2).

### Example 10. Binding Activity Verification of Anti-5T4 Antibody to NCI-H226 Cells, NCI-H2030 Cells Or HCC827 Cells

The binding activity of anti-5T4 antibody 32G1 to NCI-H226 cells (ATCC, Cat #: CRL-5826), NCI-H2030 cells or HCC827 cells (ATCC, Cat #: CRL-2868) were verified by flow cytometry.

NCI-H226 cells, NCI-H2030 cells or HCC827 cells were plated in a 96-well plate at a density of 2×10⁵ cells/well. Serially diluted purified anti-5T4 antibody 32G1 or the positive control PF06263507 analog-SI (maximum concentration: 20 μg/mL, 2-fold dilutions, 12 gradients) were added to each well and were incubated at 4° C. for 30 minutes. Then, after one wash with PBS, the cells were incubated with the secondary antibody Alexa Fluor® 647 anti human IgG Fcγ (Jackson ImmunoResearch Laboratories, Inc., Cat #: 109-606-170) at 4° C. for 15 minutes before flow cytometry analysis.

SI mutations (EU numbering: S239D and I332E mutations) are introduced into the Fc region of PF06263507 analog and the resulting antibody is named as PF06263507 analog-SI.

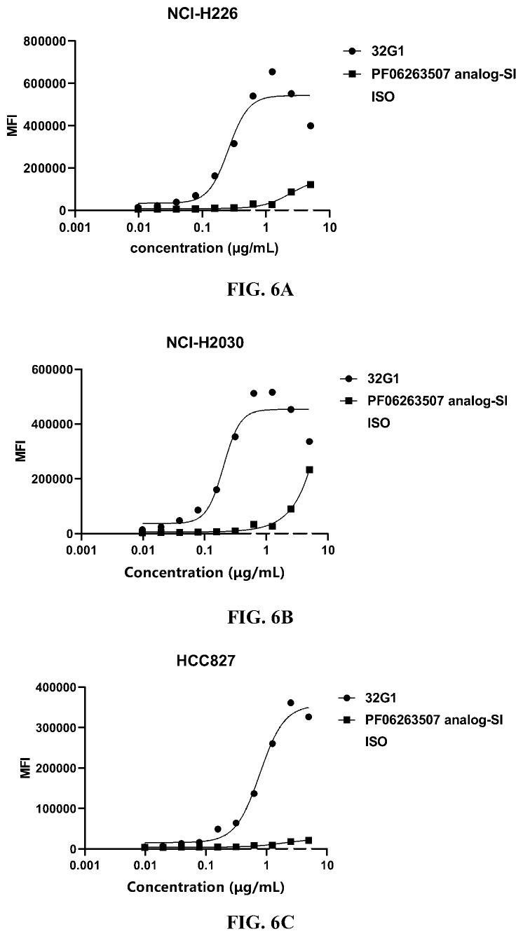

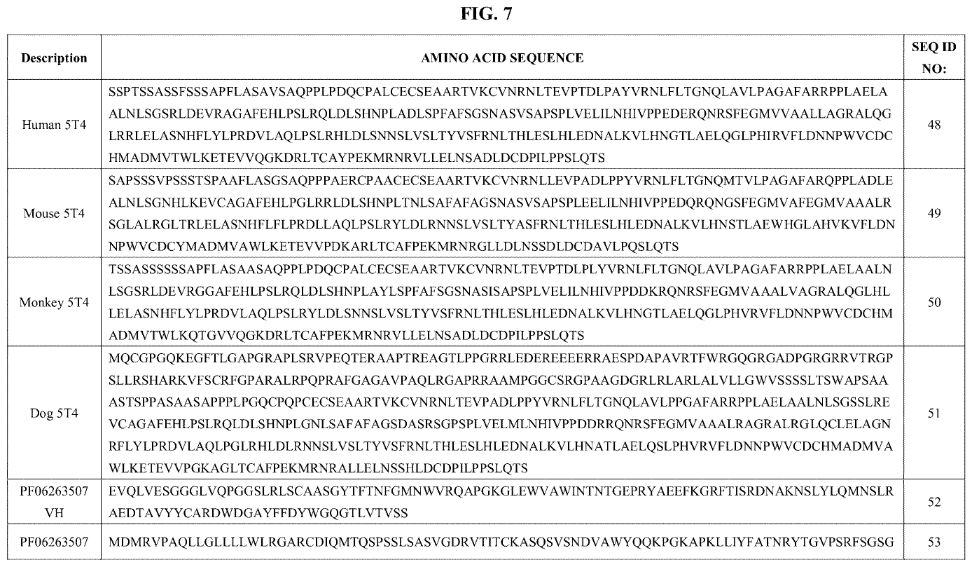

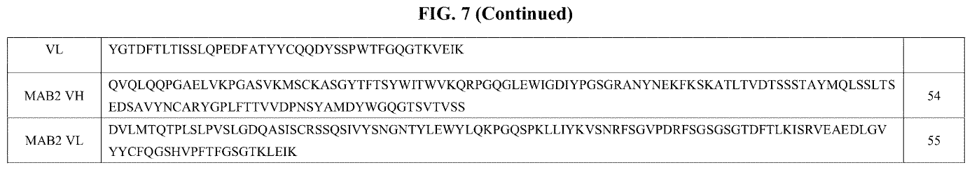

The cells were collected, and the mean fluorescence intensity (MFI) was determined. The results are shown in FIGS. 6A-6C, which showed that 32G1 exhibited higher binding activity to NCI-H226 cells, NCI-H2030 cells and HCC827 cells than that of the positive control PF06263507 analog-SI.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to 5T4 (5T4 oncofoetal antigen) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs 1, 2, and 3 amino acid sequences are one of the following:
(1) the selected VH CDRs 1, 2, 3 amino acid sequences are set forth in SEQ ID NOs: 4-6, respectively, and the selected VL CDRs 1, 2, 3 amino acid sequences are set forth in SEQ ID NOs: 1-3, respectively;
(2) the selected VH CDRs 1, 2, 3 amino acid sequences are set forth in SEQ ID NOs: 7-9, respectively, and the selected VL CDRs 1, 2, 3 amino acid sequences are set forth in SEQ ID NOs: 1-3, respectively;
(3) the selected VH CDRs 1, 2, 3 amino acid sequences are set forth in SEQ ID NOs: 10-12, respectively, and the selected VL CDRs 1, 2, 3 amino acid sequences are set forth in SEQ ID NOs: 1-3, respectively;
(4) the selected VH CDRs 1, 2, 3 amino acid sequences are set forth in SEQ ID NOs: 13-15, respectively, and the selected VL CDRs 1, 2, 3 amino acid sequences are set forth in SEQ ID NOs: 1-3, respectively;
(5) the selected VH CDRs 1, 2, 3 amino acid sequences are set forth in SEQ ID NOs: 16-18, respectively, and the selected VL CDRs 1, 2, 3 amino acid sequences are set forth in SEQ ID NOs: 1-3, respectively;
(6) the selected VH CDRs 1, 2, 3 amino acid sequences are set forth in SEQ ID NOs: 19-21, respectively, and the selected VL CDRs 1, 2, 3 amino acid sequences are set forth in SEQ ID NOs: 1-3, respectively;
(7) the selected VH CDRs 1, 2, 3 amino acid sequences are set forth in SEQ ID NOs: 22-24, respectively, and the selected VL CDRs 1, 2, 3 amino acid sequences are set forth in SEQ ID NOs: 1-3, respectively;
(8) the selected VH CDRs 1, 2, 3 amino acid sequences are set forth in SEQ ID NOs: 25-27, respectively, and the selected VL CDRs 1, 2, 3 amino acid sequences are set forth in SEQ ID NOs: 1-3, respectively;
(9) the selected VH CDRs 1, 2, 3 amino acid sequences are set forth in SEQ ID NOs: 28-30, respectively, and the selected VL CDRs 1, 2, 3 amino acid sequences are set forth in SEQ ID NOs: 1-3, respectively;
(10) the selected VH CDRs 1, 2, 3 amino acid sequences are set forth in SEQ ID NOs: 31-33, respectively, and the selected VL CDRs 1, 2, 3 amino acid sequences are set forth in SEQ ID NOs: 1-3, respectively;
(11) the selected VH CDRs 1, 2, 3 amino acid sequences are set forth in SEQ ID NOs: 34-36, respectively, and the selected VL CDRs 1, 2, 3 amino acid sequences are set forth in SEQ ID NOs: 1-3, respectively; and
(12) the selected VH CDRs 1, 2, 3 amino acid sequences are set forth in SEQ ID NOs: 37-39, respectively, and the selected VL CDRs 1, 2, 3 amino acid sequences are set forth in SEQ ID NOs: 1-3, respectively.

2. The antibody or antigen-binding fragment thereof of claim 1, wherein the VH comprises CDRs 1, 2, 3 with the amino acid sequences set forth in SEQ ID NOs: 4-6, respectively, and the VL comprises CDRs 1, 2, 3 with the amino acid sequences set forth in SEQ ID NOs: 1-3, respectively, according to Kabat definition.

3. The antibody or antigen-binding fragment thereof of claim 1, wherein the VH comprises CDRs 1, 2, 3 with the amino acid sequences set forth in SEQ ID NOs: 7-9, respectively, and the VL comprises CDRs 1, 2, 3 with the amino acid sequences set forth in SEQ ID NOs: 1-3, respectively, according to Kabat definition.

4. The antibody or antigen-binding fragment thereof of claim 1, wherein the VH comprises CDRs 1, 2, 3 with the amino acid sequences set forth in SEQ ID NOs: 10-12, respectively, and the VL comprises CDRs 1, 2, 3 with the amino acid sequences set forth in SEQ ID NOs: 1-3, respectively, according to Kabat definition.

5. The antibody or antigen-binding fragment thereof of claim 1, wherein the VH comprises CDRs 1, 2, 3 with the amino acid sequences set forth in SEQ ID NOs: 13-15, respectively, and the VL comprises CDRs 1, 2, 3 with the amino acid sequences set forth in SEQ ID NOs: 1-3, respectively, according to Kabat definition.

6. The antibody or antigen-binding fragment thereof of claim 1, wherein the VH comprises CDRs 1, 2, 3 with the amino acid sequences set forth in SEQ ID NOs: 16-18, respectively, and the VL comprises CDRs 1, 2, 3 with the amino acid sequences set forth in SEQ ID NOs: 1-3, respectively, according to Kabat definition.

7. The antibody or antigen-binding fragment thereof of claim 1, wherein the VH comprises CDRs 1, 2, 3 with the amino acid sequences set forth in SEQ ID NOs: 19-21, respectively, and the VL comprises CDRs 1, 2, 3 with the amino acid sequences set forth in SEQ ID NOs: 1-3, respectively, according to Kabat definition.

8. The antibody or antigen-binding fragment thereof of claim 1, wherein the VH comprises CDRs 1, 2, 3 with the amino acid sequences set forth in SEQ ID NOs: 22-24, respectively, and the VL comprises CDRs 1, 2, 3 with the amino acid sequences set forth in SEQ ID NOs: 1-3, respectively, according to Chothia definition.

9. The antibody or antigen-binding fragment thereof of claim 1, wherein the VH comprises CDRs 1, 2, 3 with the amino acid sequences set forth in SEQ ID NOs: 25-27, respectively, and the VL comprises CDRs 1, 2, 3 with the amino acid sequences set forth in SEQ ID NOs: 1-3, respectively, according to Chothia definition.

10. The antibody or antigen-binding fragment thereof of claim 1, wherein the VH comprises CDRs 1, 2, 3 with the amino acid sequences set forth in SEQ ID NOs: 28-30, respectively, and the VL comprises CDRs 1, 2, 3 with the amino acid sequences set forth in SEQ ID NOs: 1-3, respectively, according to Chothia definition.

11. The antibody or antigen-binding fragment thereof of claim 1, wherein the VH comprises CDRs 1, 2, 3 with the amino acid sequences set forth in SEQ ID NOs: 31-33, respectively, and the VL comprises CDRs 1, 2, 3 with the amino acid sequences set forth in SEQ ID NOs: 1-3, respectively, according to Chothia definition.

12. The antibody or antigen-binding fragment thereof of claim 1, wherein the VH comprises CDRs 1, 2, 3 with the amino acid sequences set forth in SEQ ID NOs: 34-36, respectively, and the VL comprises CDRs 1, 2, 3 with the amino acid sequences set forth in SEQ ID NOs: 1-3, respectively, according to Chothia definition.

13. The antibody or antigen-binding fragment thereof of claim 1, wherein the VH comprises CDRs 1, 2, 3 with the amino acid sequences set forth in SEQ ID NOs: 37-39, respectively, and the VL comprises CDRs 1, 2, 3 with the amino acid sequences set forth in SEQ ID NOs: 1-3, respectively, according to Chothia definition.

14. The antibody or antigen-binding fragment thereof of any one of claims 1-13, wherein the antibody or antigen-binding fragment thereof specifically binds to human, mouse, monkey, or dog 5T4.

15. The antibody or antigen-binding fragment thereof of any one of claims 1-14, wherein the antibody or antigen-binding fragment thereof is a human or humanized antibody or antigen-binding fragment thereof, a single-chain variable fragment (scFv), a one-armed antibody, and/or a multi-specific antibody (e.g., a bispecific antibody).

16. The antibody or antigen-binding fragment thereof of any one of claims 1-15, wherein the antibody or antigen-binding fragment thereof is a human IgG1 antibody or antigen-binding fragment thereof or a human IgG4 antibody or antigen-binding fragment thereof.

17. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 4-6, respectively, and wherein the VH, when paired with a light chain variable region (VL) comprising the amino acid sequence set forth in SEQ ID NO: 40 binds to 5T4;
(2) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 41 binds to 5T4;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7-9, respectively, and wherein the VH, when paired with a light chain variable region (VL) comprising the amino acid sequence set forth in SEQ ID NO: 40 binds to 5T4;
(4) an immunoglobulin light chain or a fragment thereof comprising a VL comprising complementarity determining regions (CDRs) 1, 2, and 3 comprising the amino acid sequences set forth in SEQ ID NOs: 1-3, respectively, and wherein the VL, when paired with a VH comprising the amino acid sequence set forth in SEQ ID NO: 42 binds to 5T4;
(5) an immunoglobulin heavy chain or a fragment thereof comprising a VH comprising complementarity determining regions (CDRs) 1, 2, and 3 comprising the amino acid sequences set forth in SEQ ID NOs: 10-12, respectively, and wherein the VH, when paired with a light chain variable region (VL) comprising the amino acid sequence set forth in SEQ ID NO: 40 binds to 5T4;
(6) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 43 binds to 5T4;
(7) an immunoglobulin heavy chain or a fragment thereof comprising a VH comprising complementarity determining regions (CDRs) 1, 2, and 3 comprising the amino acid sequences set forth in SEQ ID NOs: 13-15, respectively, and wherein the VH, when paired with a light chain variable region (VL) comprising the amino acid sequence set forth in SEQ ID NO: 40 binds to 5T4;
(8) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 44 binds to 5T4;
(9) an immunoglobulin heavy chain or a fragment thereof comprising a VH comprising complementarity determining regions (CDRs) 1, 2, and 3 comprising the amino acid sequences set forth in SEQ ID NOs: 16-18, respectively, and wherein the VH, when paired with a light chain variable region (VL) comprising the amino acid sequence set forth in SEQ ID NO: 40 binds to 5T4;
(10) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 45 binds to 5T4;
(11) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 46 binds to 5T4;
(12) an immunoglobulin heavy chain or a fragment thereof comprising a VH comprising complementarity determining regions (CDRs) 1, 2, and 3 comprising the amino acid sequences set forth in SEQ ID NOs: 19-21, respectively, and wherein the VH, when paired with a light chain variable region (VL) comprising the amino acid sequence set forth in SEQ ID NO: 40 binds to 5T4;
(13) an immunoglobulin light chain or a fragment thereof comprising a VL comprising CDRs 1, 2, and 3 comprising the amino acid sequences set forth in SEQ ID NOs: 1-3, respectively, and wherein the VL, when paired with a VH comprising the amino acid sequence set forth in SEQ ID NO: 47 binds to 5T4;
(14) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 22-24, respectively, and wherein the VH, when paired with a light chain variable region (VL) comprising the amino acid sequence set forth in SEQ ID NO: 40 binds to 5T4;
(15) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 25-27, respectively, and wherein the VH, when paired with a light chain variable region (VL) comprising the amino acid sequence set forth in SEQ ID NO: 40 binds to 5T4;
(16) an immunoglobulin heavy chain or a fragment thereof comprising a VH comprising complementarity determining regions (CDRs) 1, 2, and 3 comprising the amino acid sequences set forth in SEQ ID NOs: 28-30, respectively, and wherein the VH, when paired with a light chain variable region (VL) comprising the amino acid sequence set forth in SEQ ID NO: 40 binds to 5T4;
(17) an immunoglobulin heavy chain or a fragment thereof comprising a VH comprising complementarity determining regions (CDRs) 1, 2, and 3 comprising the amino acid sequences set forth in SEQ ID NOs: 31-33, respectively, and wherein the VH, when paired with a light chain variable region (VL) comprising the amino acid sequence set forth in SEQ ID NO: 40 binds to 5T4;
(18) an immunoglobulin heavy chain or a fragment thereof comprising a VH comprising complementarity determining regions (CDRs) 1, 2, and 3 comprising the amino acid sequences set forth in SEQ ID NOs: 34-36, respectively, and wherein the VH, when paired with a light chain variable region (VL) comprising the amino acid sequence set forth in SEQ ID NO: 40 binds to 5T4; or
(19) an immunoglobulin heavy chain or a fragment thereof comprising a VH comprising complementarity determining regions (CDRs) 1, 2, and 3 comprising the amino acid sequences set forth in SEQ ID NOs: 37-39, respectively, and wherein the VH, when paired with a light chain variable region (VL) comprising the amino acid sequence set forth in SEQ ID NO: 40 binds to 5T4.

18. The nucleic acid of claim 17, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 1-3, respectively.

19. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 4-6, respectively.

20. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7-9, respectively.

21. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 10-12, respectively.

22. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13-15, respectively.

23. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 16-18, respectively.

24. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 19-21, respectively.

25. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 22-24, respectively.

26. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 25-27, respectively.

27. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 28-30, respectively.

28. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 31-33, respectively.

29. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 34-36, respectively.

30. The nucleic acid of claim 17, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 37-39, respectively.

31. The nucleic acid of any one of claims 17-30, wherein the VH when paired with a VL specifically binds to human, mouse, monkey, or dog 5T4, or the VL when paired with a VH specifically binds to human, mouse, monkey, or dog 5T4.

32. The nucleic acid of any one of claims 17-31, wherein the immunoglobulin heavy chain or the fragment thereof is a human or humanized immunoglobulin heavy chain or a fragment thereof (e.g., a human IgG1 heavy chain or a fragment thereof; or a human IgG4 heavy chain or a fragment thereof), and the immunoglobulin light chain or the fragment thereof is a human or humanized immunoglobulin light chain or a fragment thereof.

33. The nucleic acid of any one of claims 17-32, wherein the nucleic acid encodes a single-chain variable fragment (scFv), a one-armed antibody, a multi-specific antibody (e.g., a bispecific antibody), or a chimeric antigen receptor (CAR).

34. The nucleic acid of any one of claims 17-33, wherein the nucleic acid is cDNA.

35. A vector comprising one or more of the nucleic acids of any one of claims 17-34.

36. A vector comprising two of the nucleic acids of any one of claims 17-34, wherein the vector encodes the VL region and the VH region that together bind to 5T4.

37. A pair of vectors, wherein each vector comprises one of the nucleic acids of any one of claims 17-34, wherein together the pair of vectors encodes the VL region and the VH region that together bind to 5T4.

38. A cell comprising the vector of claim 35 or 36, or the pair of vectors of claim 38.

39. The cell of claim 38, wherein the cell is a CHO cell.

40. A cell comprising one or more of the nucleic acids of any one of claims 17-34.

41. A cell comprising two of the nucleic acids of any one of claims 17-34.

42. The cell of claim 41, wherein the two nucleic acids together encode the VL region and the VH region that together bind to 5T4.

43. A method of producing an antibody or an antigen-binding fragment thereof, the method comprising
(a) culturing the cell of any one of claims 39-43 under conditions sufficient for the cell to produce the antibody or the antigen-binding fragment; and
(b) collecting the antibody or the antigen-binding fragment produced by the cell.

44. An antibody or antigen-binding fragment thereof that binds to 5T4 comprising
a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 41, and the selected VL sequence is SEQ ID NO: 40;
(2) the selected VH sequence is SEQ ID NO: 42, and the selected VL sequence is SEQ ID NO: 40;
(3) the selected VH sequence is SEQ ID NO: 43, and the selected VL sequence is SEQ ID NO: 40;
(4) the selected VH sequence is SEQ ID NO: 44, and the selected VL sequence is SEQ ID NO: 40;
(5) the selected VH sequence is SEQ ID NO: 45, and the selected VL sequence is SEQ ID NO: 40;
(6) the selected VH sequence is SEQ ID NO: 46, and the selected VL sequence is SEQ ID NO: 40; and
(7) the selected VH sequence is SEQ ID NO: 47, and the selected VL sequence is SEQ ID NO: 40.

45. The antibody or antigen-binding fragment thereof of claim 44, wherein the VH comprises the sequence of SEQ ID NO: 41 and the VL comprises the sequence of SEQ ID NO: 40.

46. The antibody or antigen-binding fragment thereof of claim 44, wherein the VH comprises the sequence of SEQ ID NO: 42 and the VL comprises the sequence of SEQ ID NO: 40.

47. The antibody or antigen-binding fragment thereof of claim 44, wherein the VH comprises the sequence of SEQ ID NO: 43 and the VL comprises the sequence of SEQ ID NO: 40.

48. The antibody or antigen-binding fragment thereof of claim 44, wherein the VH comprises the sequence of SEQ ID NO: 44 and the VL comprises the sequence of SEQ ID NO: 40.

49. The antibody or antigen-binding fragment thereof of claim 44, wherein the VH comprises the sequence of SEQ ID NO: 45 and the VL comprises the sequence of SEQ ID NO: 40.

50. The antibody or antigen-binding fragment thereof of claim 44, wherein the VH comprises the sequence of SEQ ID NO: 46 and the VL comprises the sequence of SEQ ID NO: 40.

51. The antibody or antigen-binding fragment thereof of claim 44, wherein the VH comprises the sequence of SEQ ID NO: 47 and the VL comprises the sequence of SEQ ID NO: 40.

52. An antibody or antigen-binding fragment thereof that binds to 5T4 comprising
a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 41, and the selected VL sequence is SEQ ID NO: 40;
(2) the selected VH sequence is SEQ ID NO: 42, and the selected VL sequence is SEQ ID NO: 40;
(3) the selected VH sequence is SEQ ID NO: 43, and the selected VL sequence is SEQ ID NO: 40;
(4) the selected VH sequence is SEQ ID NO: 44, and the selected VL sequence is SEQ ID NO: 40;
(5) the selected VH sequence is SEQ ID NO: 45, and the selected VL sequence is SEQ ID NO: 40;
(6) the selected VH sequence is SEQ ID NO: 46, and the selected VL sequence is SEQ ID NO: 40; and
(7) the selected VH sequence is SEQ ID NO: 47, and the selected VL sequence is SEQ ID NO: 40.

53. The antibody or antigen-binding fragment thereof of any one of claims 44-52, wherein the antibody or antigen-binding fragment thereof specifically binds to human, mouse, monkey, or dog 5T4.

54. The antibody or antigen-binding fragment thereof of any one of claims 44-53, wherein the antibody or antigen-binding fragment thereof is a human or humanized antibody or antigen-binding fragment thereof, a single-chain variable fragment (scFv), a one-armed antibody, and/or a multi-specific antibody (e.g., a bispecific antibody).

55. The antibody or antigen-binding fragment thereof of any one of claims 44-54, wherein the antibody or antigen-binding fragment is a human IgG1 antibody or antigen-binding fragment thereof or a human IgG4 antibody or antigen-binding fragment thereof.

56. An antibody or antigen-binding fragment thereof that cross-competes with the antibody or antigen-binding fragment thereof of any one of claims 1-16 and 44-55.

57. The antibody or antigen-binding fragment thereof of any one of claims 1-16 and 44-56, wherein the antibody or antigen-binding fragment thereof comprises a fragment crystallizable region (Fc region).

58. The antibody or antigen-binding fragment thereof of claim 57, wherein the Fc region has increased complement-dependent cytotoxicity (CDC) or antibody-dependent cellular cytotoxicity (ADCC).

59. A chimeric antigen receptor (CAR) comprising the antibody or antigen-binding fragment thereof of any one of claims 1-16 and 44-56.

60. An antibody-drug conjugate comprising the antibody or antigen-binding fragment thereof of any one of claims 1-16 and 44-58 covalently bound to a therapeutic agent.

61. The antibody drug conjugate of claim 60, wherein the therapeutic agent is a cytotoxic or cytostatic agent.

62. A method of treating a subject having cancer, the method comprising administering a therapeutically effective amount of a composition comprising the antibody or antigen-binding fragment thereof of any one of claims 1-16 and 44-58, the CAR of claim 59 or the antibody-drug conjugate of claim 60 or 61, to the subject.

63. The method of claim 62, wherein the subject has a solid tumor.

64. The method of claim 62, wherein the cancer is colorectal cancer, gastric cancer, ovarian cancer, lung cancer, head and neck cancer, or pancreatic cancer.

65. The method of claim 62, wherein the subject is further treated with an effective amount of an anti-4-1BB antibody, an anti-OX40 antibody, an anti-PD-1 antibody, an anti-CTLA4 antibody, an anti-CD40 antibody, or an anti-PD-L1 antibody.

66. A method of decreasing the rate of tumor growth, the method comprising contacting a tumor cell with an effective amount of a composition comprising an antibody or antigen-binding fragment thereof of any one of claims 1-16 and 44-58, the CAR of claim 59, or the antibody-drug conjugate of claim 60 or 61.

67. A method of killing a tumor cell, the method comprising contacting a tumor cell with an effective amount of a composition comprising the antibody or antigen-binding fragment thereof of any one of claims 1-16 and 44-58, the CAR of claim 59, or the antibody-drug conjugate of claim 60 or 61.

68. A method of increasing immune response in a subject, the method comprising administering to the subject an effective amount of a composition comprising the antibody or antigen-binding fragment thereof of any one of claims 1-16 and 44-58, the CAR of claim 59, or the antibody-drug conjugate of claim 60 or 61.

69. A pharmaceutical composition comprising the antibody or antigen-binding fragment thereof of any one of claims 1-16 and 44-58, and a pharmaceutically acceptable carrier.

70. A pharmaceutical composition comprising the antibody drug conjugate of claim 60 or 61, and a pharmaceutically acceptable carrier.

71. The antibody-drug conjugate of claim 60 or 61, wherein the drug-to-antibody ratio (DAR) is about 4.


SEQUENCE LISTING (55 sequences)

SEQ ID NO: 1 - protein, 11 aa, synthetic construct
RASQSVSSYLA

SEQ ID NO: 2 - protein, 7 aa, synthetic construct
DASNRAT

SEQ ID NO: 3 - protein, 9 aa, synthetic construct
QQRSNWPPT

SEQ ID NO: 4 - protein, 5 aa, synthetic construct
SYWMH

SEQ ID NO: 5 - protein, 17 aa, synthetic construct
RINFDGSSITYADSVKG

SEQ ID NO: 6 - protein, 17 aa, synthetic construct
PLTYYEILTGFSQYFDF

SEQ ID NO: 7 - protein, 5 aa, synthetic construct
SDYMS

SEQ ID NO: 8 - protein, 16 aa, synthetic construct
IIYSGGSTFYADSVKG

SEQ ID NO: 9 - protein, 17 aa, synthetic construct
DRDCHGWGSYYNRWFDP

SEQ ID NO: 10 - protein, 5 aa, synthetic construct
DYYMN

SEQ ID NO: 11 - protein, 17 aa, synthetic construct
YVGTESIYTYYADTVKG

SEQ ID NO: 12 - protein, 11 aa, synthetic construct
ARDYNLYGMDV

SEQ ID NO: 13 - protein, 7 aa, synthetic construct
SSGHYWG

SEQ ID NO: 14 - protein, 16 aa, synthetic construct
SIYFSGNTNYNPSLKS

SEQ ID NO: 15 - protein, 15 aa, synthetic construct
RNGRYCSTISCYDDY

SEQ ID NO: 16 - protein, 5 aa, synthetic construct
SNDWS

SEQ ID NO: 17 - protein, 16 aa, synthetic construct
EISYSGITNYNPSLKS

SEQ ID NO: 18 - protein, 14 aa, synthetic construct
AGGSSGWYDYWFDP

SEQ ID NO: 19 - protein, 5 aa, synthetic construct
RNAMS

SEQ ID NO: 20 - protein, 17 aa, synthetic construct
AISGSGGSTHDADSVKG

SEQ ID NO: 21 - protein, 16 aa, synthetic construct
DGYSSLWDYHYYGMDV

SEQ ID NO: 22 - protein, 7 aa, synthetic construct
GFTFKSY

SEQ ID NO: 23 - protein, 6 aa, synthetic construct
NFDGSS

SEQ ID NO: 24 - protein, 17 aa, synthetic construct
PLTYYEILTGFSQYFDF

SEQ ID NO: 25 - protein, 7 aa, synthetic construct
GFTVSSD

SEQ ID NO: 26 - protein, 5 aa, synthetic construct
YSGGS

SEQ ID NO: 27 - protein, 17 aa, synthetic construct
DRDCHGWGSYYNRWFDP

SEQ ID NO: 28 - protein, 7 aa, synthetic construct
GFTFSDY

SEQ ID NO: 29 - protein, 6 aa, synthetic construct
GTESIY

SEQ ID NO: 30 - protein, 11 aa, synthetic construct
ARDYNLYGMDV

SEQ ID NO: 31 - protein, 9 aa, synthetic construct
GASINSSGH

SEQ ID NO: 32 - protein, 5 aa, synthetic construct
YFSGN

SEQ ID NO: 33 - protein, 15 aa, synthetic construct
RNGRYCSTISCYDDY

SEQ ID NO: 34 - protein, 7 aa, synthetic construct
GGSISSN

SEQ ID NO: 35 - protein, 5 aa, synthetic construct
SYSGI

SEQ ID NO: 36 - protein, 14 aa, synthetic construct
AGGSSGWYDYWFDP

SEQ ID NO: 37 - protein, 7 aa, synthetic construct
GFTFSRN

SEQ ID NO: 38 - protein, 6 aa, synthetic construct
SGSGGS

SEQ ID NO: 39 - protein, 16 aa, synthetic construct
DGYSSLWDYHYYGMDV

SEQ ID NO: 40 - protein, 107 aa, synthetic construct [sequence omitted]

SEQ ID NO: 41 - protein, 126 aa, synthetic construct
QVQLVQSGGGLVQPGGSLRLSCAASGFTFKSYWMHWVRQAPGKGLEWVSRINFDGSSITYADSVKGRFTISRDNAKNTLYLQMNSLSAEDTAVYYCARPLTYYEILTGFSQYFDFWGQGTLVTVSS

SEQ ID NO: 42 - protein, 125 aa, synthetic construct
QVQLVQSGGGLVQPGGSLRLSCVVSGFTVSSDYMSWVRQAPGKGLEWVSIIYSGGSTFYADSVKGRFTISRDNSKNTLFLQMNSLRAEDTAVYYCARDRDCHGWGSYYNRWFDPWGQGTLVTVSS

SEQ ID NO: 43 - protein, 120 aa, synthetic construct
QVQLVQSGGGLVKPGGSLRLSCVASGFTFSDYYMNWVRQAPGKGLEWISYVGTESIYTYYADTVKGRFTISRDNAKNSLYLQMNSLRAEDTALYYCARARDYNLYGMDVWGQGTTVTVSS

SEQ ID NO: 44 - protein, 125 aa, synthetic construct
QVQLQESGPGLLQPSETLSLTCFVSGASINSSGHYWGWIRQPPGKGLEWIGSIYFSGNTNYNPSLKSRVTIAVDTSKNQFSLKLSSVTAADTAVYYCVRRNGRYCSTISCYDDYWGQGTLVTVSS

SEQ ID NO: 45 - protein, 122 aa, synthetic construct
QVQLQESGPRLVKPSETLSLTCTVSGGSISSNDWSWIRQPPGKGLEWIGEISYSGITNYNPSLKSRVSISVETSKNHFSLNLSSVTAADTAVYYCATAGGSSGWYDYWFDPWGQGTLVTVSS

SEQ ID NO: 46 - protein, 122 aa, synthetic construct
QVQLQESGPRLVKPSETLSLTCTVSGGSISSNDWSWIRQPPGKGLEWIGEISYSGITNYNPSLKSRVSISVETSKNHFSLKLSSVTAADTAVYYCATAGGSSGWYDYWFDPWGQGTLVTVSS

SEQ ID NO: 47 - protein, 125 aa, synthetic construct
QVQLVQSGGGLVQPGGSLRLSCAASGFTFSRNAMSWVRQAPGKGLEWVSAISGSGGSTHDADSVKGRFTISRDNSKNTLYLEMNSLRAEDTAVYYCAKDGYSSLWDYHYYGMDVWGQGTTVTVSS

SEQ ID NO: 48 - protein, 324 aa, Homo sapiens
SSPTSSASSFSSSAPFLASAVSAQPPLPDQCPALCECSEAARTVKCVNRNLTEVPTDLPAYVRNLFLTGNQLAVLPAGAFARRPPLAELAALNLSGSRLDEVRAGAFEHLPSLRQLDLSHNPLADLSPFAFSGSNASVSAPSPLVELILNHIVPPEDERQNRSFEGMVVAALLAGRALQGLRRLELASNHFLYLPRDVLAQLPSLRHLDLSNNSLVSLTYVSFRNLTHLESLHLEDNALKVLHNGTLAELQGLPHIRVFLDNNPWVCDCHMADMVTWLKETEVVQGKDRLTCAYPEKMRNRVLLELNSADLDCDPILPPSLQTS

SEQ ID NO: 49 - protein, 330 aa, Mus musculus
SAPSSSVPSSSTSPAAFLASGSAQPPPAERCPAACECSEAARTVKCVNRNLLEVPADLPPYVRNLFLTGNQMTVLPAGAFARQPPLADLEALNLSGNHLKEVCAGAFEHLPGLRRLDLSHNPLTNLSAFAFAGSNASVSAPSPLEELILNHIVPPEDQRQNGSFEGMVAFEGMVAAALRSGLALRGLTRLELASNHFLFLPRDLLAQLPSLRYLDLRNNSLVSLTYASFRNLTHLESLHLEDNALKVLHNSTLAEWHGLAHVKVFLDNNPWVCDCYMADMVAWLKETEVVPDKARLTCAFPEKMRNRGLLDLNSSDLDCDAVLPQSLQTS

SEQ ID NO: 50 - protein, 321 aa, Macaca fascicularis
TSSASSSSSSAPFLASAASAQPPLPDQCPALCECSEAARTVKCVNRNLTEVPTDLPLYVRNLFLTGNQLAVLPAGAFARRPPLAELAALNLSGSRLDEVRGGAFEHLPSLRQLDLSHNPLAYLSPFAFSGSNASISAPSPLVELILNHIVPPDDKRQNRSFEGMVAAALVAGRALQGLHLLELASNHFLYLPRDVLAQLPSLRYLDLSNNSLVSLTYVSFRNLTHLESLHLEDNALKVLHNGTLAELQGLPHVRVFLDNNPWVCDCHMADMVTWLKQTGVVQGKDRLTCAFPEKMRNRVLLELNSADLDCDPILPPSLQTS

SEQ ID NO: 51 - protein, 488 aa, Canis lupus
MQCGPGQKEGFTLGAPGRAPLSRVPEQTERAAPTREAGTLPPGRRLEDEREEEERRAESPDAPAVRTFWRGQGRGADPGRGRRVTRGPSLLRSHARKVFSCRFGPARALRPQPRAFGAGAVPAQLRGAPRRAAMPGGCSRGPAAGDGRLRLARLALVLLGWVSSSSLTSWAPSAAASTSPPASAASAPPPLPGQCPQPCECSEAARTVKCVNRNLTEVPADLPPYVRNLFLTGNQLAVLPPGAFARRPPLAELAALNLSGSSLREVCAGAFEHLPSLRQLDLSHNPLGNLSAFAFAGSDASRSGPSPLVELMLNHIVPPDDRRQNRSFEGMVAAALRAGRALRGLQCLELAGNRFLYLPRDVLAQLPGLRHLDLRNNSLVSLTYVSFRNLTHLESLHLEDNALKVLHNATLAELQSLPHVRVFLDNNPWVCDCHMADMVAWLKETEVVPGKAGLTCAFPEKMRNRALLELNSSHLDCDPILPPSLQTS

SEQ ID NO: 52 - protein, 119 aa, synthetic construct
EVQLVESGGGLVQPGGSLRLSCAASGYTFTNFGMNWVRQAPGKGLEWVAWINTNTGEPRYAEEFKGRFTISRDNAKNSLYLQMNSLRAEDTAVYYCARDWDGAYFFDYWGQGTLVTVSS

SEQ ID NO: 53 - protein, 129 aa, synthetic construct
MDMRVPAQLLGLLLLWLRGARCDIQMTQSPSSLSASVGDRVTITCKASQSVSNDVAWYQQKPGKAPKLLIYFATNRYTGVPSRFSGSGYGTDFTLTISSLQPEDFATYYCQQDYSSPWTFGQGTKVEIK

SEQ ID NO: 54 - protein, 127 aa, synthetic construct
QVQLQQPGAELVKPGASVKMSCKASGYTFTSYWITWVKQRPGQGLEWIGDIYPGSGRANYNEKFKSKATLTVDTSSSTAYMQLSSLTSEDSAVYNCARYGPLFTTVVDPNSYAMDYWGQGTSVTVSS

SEQ ID NO: 55 - protein, 112 aa, synthetic construct
DVLMTQTPLSLPVSLGDQASISCRSSQSIVYSNGNTYLEWYLQKPGQSPKLLIYKVSNRFSGVPDRFSGSGSGTDFTLKISRVEAEDLGVYYCFQGSHVPFTFGSGTKLEIK